(19) United States
(12) Patent Application Publication
(10) Pub. No.: US 2008/0293785 A1
(43) Pub. Date: Nov. 27, 2008

(54) SUBSTITUTED BENZOTHIAZOLE KINASE INHIBITORS

(76) Inventors: Peter J. Connolly, New Providence, NJ (US); Stuart L. Emanuel, Doylestown, PA (US); Shenlin Huang, San Diego, CA (US); Ignatius J. Turchi, Yardley, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/733,278

(22) Filed: Apr. 10, 2007

Related U.S. Application Data
(60) Provisional application No. 60/791,035, filed on Apr. 11, 2006.

(51) Int. Cl. A61K 31/428; C07D 277/82; A61P 19/02; A61P 3/10; A61P 7/12; A61P 1/00; A61P 25/00; A61P 9/00; A61P 35/00; A61P 31/00

(52) U.S. Cl. 514/367; 548/164

(57) ABSTRACT

The present invention is directed to substituted benzothiazole compounds of formula (I):

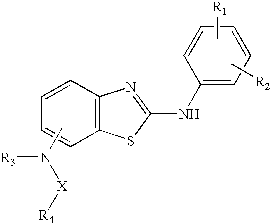

and forms thereof, their synthesis and use for treating a chronic or acute protein kinase mediated disease, disorder or condition.

### CROSS REFERENCE TO RELATED APPLICATIONS

This present application claims benefit of U.S. Provisional Patent Application Ser. No. 60/791,035, filed Apr. 11, 2006, which is incorporated herein by reference in its entirety and for all purposes.

### FIELD OF THE INVENTION

The present invention is in the area of substituted benzothiazole compounds and forms thereof and methods of preparation and use thereof as kinase inhibitors.

### BACKGROUND OF THE INVENTION

In general, protein kinases are the largest set of structurally related phosphoryl transferases, have highly conserved structures and catalytic functions and may be categorized into families by the substrates they phosphorylate (e.g., protein-tyrosine, protein-serine/threonine, histidine and the like) and are responsible for the control of a wide variety of cellular signal transduction processes.

Examples of protein-tyrosine kinases include, but are not limited to, Irk, IGFR-1, Zap-70, Bmx, Btk, CHK (Csk homologous kinase), CSK (C-terminal Src Kinase), Itk-1, Src (c-Src, Lyn, Fyn, Lck, Syk, Hck, Yes, Blk, Fgr and Frk), Tec, Txk/Rlk, Abl, EGFR (EGFR-1/ErbB-1, ErbB-2/NEU/HER-2, ErbB-3 and ErbB-4), FAK, FGF1R (also FGFR1 or FGR-1), FGF2R (also FGR-2), MET (also Met-1 or c-MET), PDGFR (α and β), Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, RET, TRKA, PYK2, ALK (Anaplastic Lymphoma Kinase), EPHA (1-8), EPHB (1-6), RON, Fes, Fer or EPHB4 (also EPHB4-1).

Examples of protein-serine/threonine kinases include, but are not limited to, Ark, ATM (1-3), CamK (I-IV), CamKK, Chk1 and 2 (Checkpoint kinases), CK1, CK2, Erk, IKK-I (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (α and β), PKA, P38, Erk (1-3), PKB (including all PKB subtypes) (also AKT-1, AKT-2, AKT-3 or AKT3-1), IRAK1, FRK, SGK, TAK1or Tp1-2 (also COT).

Protein kinases play very important roles in the normal regulation of cell growth. However, as a result of dysregulation of the tyrosine kinases (receptor or non-receptor) or the ligands of the receptor tyrosine kinases, signaling can become deregulated, resulting in uncontrolled cell proliferation leading to cancer or a related disease, disorder or syndrome.

Protein kinases catalyze and regulate the process of phosphorylation, whereby the kinases covalently attach phosphate groups to proteins or lipid targets in response to a variety of extracellular signals: hormones, neurotransmitters, growth and differentiation factors, cell cycle events, environmental stresses, nutritional stresses and the like.

Phosphorylation modulates or regulates a variety of cellular processes such as proliferation, growth, differentiation, metabolism, apoptosis, motility, transcription, translation and other signaling processes. Defective control of protein phosphorylation due to unregulated cellular mitosis, unregulated cell proliferation and upregulated kinase activity has been implicated in a number of diseases and disease conditions, such as osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohns disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, occular diseases, viral infections, heart disease, lung or pulmonary diseases or kidney or renal diseases. Therefore, kinase inhibitors have potential use as therapeutic agents.

The term “myasthenia gravis” means a disease having the characteristic feature of easy fatigue of certain voluntary muscle groups on repeated use. Muscles of the face or upper trunk are especially likely to be affected. In most and perhaps all cases, the disease is due to the development of autoantibodies against the acetylcholine receptor in neuromuscular junctions. Immunization of animals with this receptor protein leads to a disease with the features of myasthenia gravis.

In reference to “synovial pannus invasion in arthritis,” the term “pannus” means a disease whereby vascularised granulation tissue rich in fibroblasts, lymphocytes and macrophages, derived from synovial tissue, overgrows the bearing surface of the joint in rheumatoid arthritis and is associated with the breakdown of the articular surface.

The tyrosine kinases can further be categorized by whether they are receptor tyrosine kinases or non-receptor tyrosine kinases. The receptor tyrosine kinases span the cell membrane with a ligand interacting domain protruding from the cell, with a hydrophobic trans-membrane domain, and a cytoplasmic domain that contains the catalytic kinase domain and other regulatory sequences. Non-receptor tyrosine kinases are often myristylated or modified by the addition of other hydrophobic moieties that allow them to be anchored to the cell membrane.

Cyclin dependent kinases (CDK) constitute a class of enzymes that play critical roles in regulating the transitions between different phases of the cell cycle, such as the progression from a quiescent stage in G₁ (the gap between mitosis and the onset of DNA replication for a new round of cell division) to S (the period of DNA synthesis), or the progression from G₂ to M phase, in which active mitosis and cell-division occur. See, e.g., the articles compiled in Science, vol. 274 (1996), p. 1643-1677; and Ann. Rev. Cell Dev. Biol, vol. 13 (1997), pp. 261-291. CDK complexes are formed through association of a regulatory cyclin subunit (e.g., cyclin A, B1, B2, D1, D2, D3, and E) and a catalytic kinase subunit (e.g., cdc2 (CDK1), CDK2, CDK4, CDK5, and CDK6). As the name implies, the CDKs display an absolute dependence on the cyclin subunit in order to phosphorylate their target substrates, and different kinase/cyclin pairs function to regulate progression through specific portions of the cell cycle.

The D cyclins are sensitive to extracellular growth signals and become activated in response to mitogens during the G₁ phase of the cell cycle. CDK4/cyclin D plays an important role in cell cycle progression by phosphorylating, and thereby inactivating, the retinoblastoma protein (Rb). Hypophosphorylated Rb binds to a family of transcriptional regulators, but upon hyperphosphorylation of Rb by CDK4/cyclin D, these transcription factors are released to activate genes whose products are responsible for S phase progression. Rb phosphorylation and inactivation by CDK4/cyclin D permit passage of the cell beyond the restriction point of the G₁ phase, whereupon sensitivity to extracellular growth or inhibitory signals is lost and the cell is committed to cell division. During late G₁, Rb is also phosphorylated and inactivated by CDK2/cyclin E, and recent evidence indicates that CDK2/cyclin E can also regulate progression into S phase through a parallel pathway that is independent of Rb phosphorylation (see Lukas et al., “Cyclin E-induced S Phase Without Activation of the pRb/E2F Pathway,” Genes and Dev., vol. 11 (1997), pp. 1479-1492).

The progression from G₁ to S phase, accomplished by the action of CDK4/cyclin D and CDK2/cyclin E, is subject to a variety of growth regulatory mechanisms, both negative and positive. Growth stimuli, such as mitogens, caused increased synthesis of cyclin D1 and thus increased functional CDK4. By contrast, cell growth can be “reined in,” in response to DNA damage or negative growth stimuli, by the induction of endogenous inhibitory proteins. These naturally occurring protein inhibitors include p21^{WAF1/CIP1}, p27^{KIP1}, and the p16^{INK4} family, the latter of which inhibit CDK4 exclusively (see Harper, “Cyclin Dependent Kinase Inhibitors,” Cancer Surv., vol. 29 (1997), pp. 91-107). Aberrations in this control system, particularly those that affect the function of CDK4 and CKD2, are implicated in the advancement of cells to the highly proliferative state characteristic of malignancies, such as familial melanomas, esophageal carcinomas, and pancreatic cancers (see, e.g., Hall and Peters, “Genetic Alterations of Cyclins, Cyclin-Dependent Kinases, and CDK Inhibitors in Human Cancer,” Adv. Cancer Res., vol. 68 (1996), pp. 67-108; and Kamb et al., “A Cell Cycle Regulator Potentially Involved in Genesis of Many Tumor Types,” Science, vol. 264 (1994), pp. 436-440). Over-expression of cyclin D1 is linked to esophageal, breast, and squamous cell carcinomas (see, e.g., DelSal et al., “Cell Cycle and Cancer: Critical Events at the G₁ Restriction Point,” Critical Rev. Oncogenesis, vol. 71 (1996), pp. 127-142). Genes encoding the CDK4-specific inhibitors of the p16 family frequently have deletions and mutations in familial melanoma, gliomas, leukemias, sarcomas, and pancreatic, non-small cell lung, and head and neck carcinomas (see Nobori et al., “Deletions of the Cyclin-Dependent Kinase-4 Inhibitor Gene in Multiple Human Cancers,” Nature, vol. 368 (1994), pp. 753-756). Amplification and/or overexpression of cyclin E has also been observed in a wide variety of solid tumors, and elevated cyclin E levels have been correlated with poor prognosis. In addition, the cellular levels of the CDK inhibitor p27, which acts as both a substrate and inhibitor of CDK2/cyclin E, are abnormally low in breast, colon, and prostate cancers, and the expression levels of p27 are inversely correlated with the state of disease (see Loda et al., “Increased Proteasome-dependent Degradation of the Cyclin-Dependent Kinase Inhibitor p27 in Aggressive Colorectal Carcinomas,” Nature Medicine, vol. 3 (1997), pp. 231-234). The p21 protein also appear to transmit the p53 tumor-suppression signal to the CDKs; thus, the mutation of p53 in approximately 50% of all human cancers may indirectly result in deregulation of CDK activity.

In the eukaryotic cell cycle a key role is played by the cyclin dependent kinases. CDK complexes are formed via the association of a regulatory cyclin subunit and a catalytic kinase subunit. In mammalian cells, the combination of the kinase subunits (such as CDK1, CDK2, CDK4 or CDK6) with a variety of cyclin subunits (such as cyclin A, B, D1, D2, D3 or E) results in the assembly of functionally distinct kinase complexes. The coordinated activation of these complexes drives the cells through the cell cycle and ensures the fidelity of the process (Draetta, G., Trends Biochem. Sci., 1990, 15:378-382; Sherr, C. J., Cell, 1993, 73:1059-1065). Each step in the cell cycle is regulated by a distinct and specific cyclin-dependent kinase. Regulation occurs at the boundaries of the G1/S and G2/M phases, two major transition points of the cell cycle. For example, complexes of CDK4 and D-type cyclins govern the early G1 phase of the cell cycle, while the activity of the CDK2/cyclin E complex is rate limiting for the G1 to S-phase transition. The CDK2/cyclin A kinase is required for the progression through S-phase and the CDK1/cyclin B complex controls the entry into M-phase (Sherr, 1993). A key regulator of these transitions is CDK1 kinase, a universal intracellular factor which triggers the G2/M transition of the cell cycle in all organisms. Both biochemical and genetic evidence have shown that CDK1 is the primary activity required for a cell to enter mitosis in all eukaryotic cells. In late G2, it is present as an inactive complex of CDK1 and cyclin B. In M phase, it is activated and thereafter displays kinase activity. CDK1 is known to phosphorylate a number of proteins including histone H1, DNA polymerase alpha, RNA polymerase II, retinoblastoma tumor suppressor protein (RB), p53, nucleolin, cAb1 and lamin A. The kinase activity of CDK1 is required for entry of cells into mitosis, i.e., for passage from the G2 phase of the cell cycle into the M phase (Lee M. and Nurse P., Trends Genet., 1988, 4:289-90; Dunphy W. G., Brizuela L., Beach D. and Newport J., Cell, 1988, 54:423-431; Gautier J., Norbury C., Lohka M., Nurse P. and Maller J., Cell, 1988, 54:433-439; Cross F., Roberts J. and Weintraub H., Ann. Rev. Cell Biol., 1989, 5:341-395; Hunt, T. and Sherr, C., Curr. Opinion Cell Biol., 1989, 1:268-274; and, Nurse, P., Nature, 1990, 344:503-508). Therefore, using cyclin dependent kinase inhibitors for tumor therapy has the potential for inhibiting tumor growth or controlling unregulated cell proliferation.

Many conventional cytotoxic cancer therapies destroy the rapidly dividing epithelium of the hair follicle and induce alopecia (hair loss). Inhibition of cyclin dependent kinases during conventional chemotherapy may represent a therapeutic strategy for prevention of chemotherapy-induced alopecia by arresting the cell cycle and reducing the sensitivity of epithelial cells to antitumor agents (Davis S. T., et al., Prevention of chemotherapy-induced alopecia in rats by CDK inhibitors, Science, 2001, (January 5), 291, 5501, 25-6). Accordingly, to be useful in a method for the prevention of chemotherapy-induced alopecia, a CDK inhibitor compound would have to be cytostatic rather than cytotoxic and be able to hold the cell in a stationary growth phase, thus protecting a hair follicle from the cytotoxic activity of a conventional chemotherapeutic agent being administered at the same time. In this way, topical application of non-apoptotic CDK inhibitors represents a potentially useful approach for the prevention of chemotherapy-induced alopecia in cancer patients.

A second protein target that can facilitate elimination of a tumor is the tyrosine kinase vascular endothelial growth factor (VEGF) receptor. This protein is associated with both normal and pathological angiogenesis. The VEGF receptors are tripartite, consisting of an extracellular ligand-binding domain, a transmembrane domain and an intracellular tyrosine kinase domain. Presently there are two known VEGF receptors: (1) VEGF-R2 (KDR/Flk1 VEGF-R2), a receptor that mediates the biological activities of mitogenesis and proliferation of endothelial cells; and (2) VEGF-R1 (Flt1/VEGF-R1), a receptor that mediates functions such as endothelial cell adhesion. Inhibition of VEGF-R2 signalling has been shown to inhibit the process of angiogenesis. Inhibitors of this receptor are likely useful in controlling or limiting angiogenesis.

Many conventional cytotoxic cancer therapies destroy the rapidly dividing epithelium of the hair follicle and induce alopecia (hair loss). Inhibition of cyclin dependent kinases during conventional chemotherapy may represent a therapeutic strategy for prevention of chemotherapy-induced alopecia by arresting the cell cycle and reducing the sensitivity of epithelial cells to antitumor agents (Davis S. T., et al., Prevention of chemotherapy-induced alopecia in rats by CDK inhibitors, Science, 2001, (January 5), 291, 5501, 25-6). Accordingly, to be useful for such an application, a CDK inhibitor compound would have to be cytostatic, rather than cytotoxic and be able to hold the cell in a stationary growth phase which would protect it from the cytotoxic activity of a conventional chemotherapeutic agent being administered at the same time. In this way, topical application of non-apoptotic CDK inhibitors represents a potentially useful approach for the prevention of chemotherapy-induced alopecia in cancer patients.

Although coronary angioplasty is a highly effective procedure used to reduce the severity of coronary occlusion, its long-term success is limited by a high rate of restenosis. Vascular smooth muscle cell activation, migration and proliferation is largely responsible for restenosis following angioplasty (Ross, R., Nature, 1993, 362, 801-809). Recent studies have shown that CDK2 is activated very early after endothelial denudation in a rat carotid artery model of restenosis (Wei, G. L., et al., Circ. Res., 1997, 80, 418-426). Therefore, antiproliferative therapies targeted to cyclin dependent kinases or other components of the cell cycle machinery may be a suitable approach to treat these disorders. One aspect for use of the compounds of the present invention is a method for the treatment of restenosis wherein a CDK inhibitor is impregnated on the surface of an angioplasty balloon or stent, thus targeting drug delivery to the local environment where endothelial and smooth muscle cell proliferation are the leading cause of vascular occlusion following an initial angioplasty and restenosis in the area of a stent's implantation (Eric E. Brooks, Nathanael S. Gray, Alison Joly, Suresh S. Kerwar, Robert Lum, Richard L. Mackman, Thea C. Norman, Jose Rosete, Michael Rowe, Steven R. Schow, Peter G. Schultz, Xingbo Wang, Michael M. Wick and Dov Shiffman, CVT-313, a Specific and Potent Inhibitor of CDK2 That Prevents Neointimal Proliferation, J. Biol. Chem., 1997, 272(46):29207-29211).

There is a need for potent small-molecule kinase inhibitors of one or more of the CDK or VEGF kinase proteins and the like possessing anti-angiogenic, anti-hyperproliferative or anti-tumor cell proliferation activity, and as such are useful for treating a CDK or VEGF kinase receptor kinase mediated disease, disorder or condition.

### SUMMARY OF THE INVENTION

The present invention is directed to a compound of formula (I):

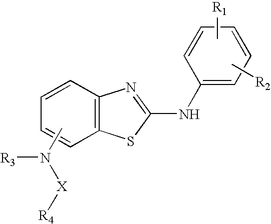

and forms thereof, wherein R₁, R₂, R₃, R₄ and X are as defined herein.

An example of the present invention includes a compound of formula (I) and forms thereof as a protein kinase inhibitor.

An example of the present invention includes a prodrug form of a compound of formula (I) and forms thereof as a protein kinase inhibitor.

An example of the present invention includes a metabolite form of a compound of formula (I) and forms thereof as a protein kinase inhibitor.

An example of the present invention includes use of a compound of formula (I) and forms thereof as an inhibitor of a protein kinase such as CDK or VEGF comprising contacting the protein kinase domain or receptor with the compound.

An example of the present invention includes the use of a compound of formula (I) and forms thereof as a pharmaceutical composition, medicine or medicament for treating a kinase mediated disease, disorder or condition.

An example of the present invention includes the use of a compound of formula (I) and forms thereof in the manufacture of a medicament for treating a kinase mediated disease, disorder or condition.

An example of the present invention includes the use of a prodrug of a compound of formula (I) and forms thereof as a pharmaceutical composition, medicine or medicament for treating a kinase mediated disease, disorder or condition.

An example of the present invention includes the use of a prodrug of a compound of formula (I) and forms thereof in the manufacture of a medicament for treating a kinase mediated disease, disorder or condition.

The present invention is further directed to a method for treating a chronic or acute protein kinase mediated disease, disorder or condition in a subject in need thereof comprising administering to the subject an effective amount of a compound of formula (I) and forms thereof.

An example of the present invention includes a method for treating a chronic or acute protein kinase mediated disease, disorder or condition in a subject in need thereof comprising administering to the subject an effective amount of a prodrug of a compound of formula (I) and forms thereof.

These and other aspects and advantages of the invention, which will become apparent in light of the detailed description below, are achieved through use of the compounds of this invention.

### DETAILED DESCRIPTION OF THE INVENTION

The present invention is directed to a compound of formula (I):

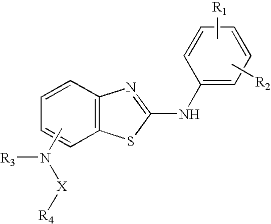

and forms thereof, wherein
R₁ is hydrogen or is selected from C₁₋₆alkyl, C₁₋₆alkoxy, amino, halogen, cyano, amino-sulfonyl, C₁₋₄alkyl-amino-sulfonyl, halo-C₁₋₄alkyl or halo-C₁₋₄alkoxy;
R₂ is hydrogen or is selected from aryl, heteroaryl, heterocyclyl or C₃₋₁₂cycloalkyl optionally substituted with one or two substituents selected from C₁₋₆alkyl, C₁₋₆alkoxy or halogen;
R₃ is hydrogen or is C₁₋₄alkyl;
X is selected from carbonyl, amino-carbonyl, oxy-carbonyl or sulfonyl; and
R₄ is selected from a C₁₋₄alkyl, aryl, aryl-C₁₋₄alkyl, heteroaryl, heteroaryl-C₁₋₄alkyl, heterocyclyl, heterocyclyl-C₁₋₄alkyl, C₃₋₁₂cycloalkyl or C₃₋₁₂cycloalkyl-C₁₋₄alkyl optionally substituted on aryl, heteroaryl, heterocyclyl or C₃₋₁₂cycloalkyl with one, two or three substituents selected from C₁₋₆alkyl, C₁₋₆alkenyl, C₁₋₆alkynyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl, heteroaryl, heterocyclyl or C₃₋₁₂cycloalkyl.

An example of a compound of formula (I) and forms thereof includes a compound wherein R₁ is hydrogen or is selected from C₁₋₆alkyl, C₁₋₆alkoxy, amino, halogen, cyano or amino-sulfonyl.

An example of a compound of formula (I) and forms thereof includes a compound wherein R₁ is hydrogen or is selected from C₁₋₆alkyl, C₁₋₆alkoxy or amino-sulfonyl.

An example of a compound of formula (I) and forms thereof includes a compound wherein R₂ is hydrogen or is selected from aryl, heteroaryl, heterocyclyl or C₃₋₁₂cycloalkyl optionally substituted with one substituent selected from C₁₋₆alkyl, C₁₋₆alkoxy or halogen.

An example of a compound of formula (I) and forms thereof includes a compound wherein R₂ is hydrogen or is heterocyclyl optionally substituted with C₁₋₆alkyl.

An example of a compound of formula (I) and forms thereof includes a compound wherein R₂ is hydrogen.

An example of a compound of formula (I) and forms thereof includes a compound wherein R₂ is heterocyclyl optionally substituted with C₁₋₆alkyl.

An example of a compound of formula (I) and forms thereof includes a compound wherein R₂ is piperazinyl optionally substituted with C₁₋₆alkyl.

An example of a compound of formula (I) and forms thereof includes a compound wherein R₃ is hydrogen.

An example of a compound of formula (I) and forms thereof includes a compound wherein R₃ is C₁₋₄alkyl.

An example of a compound of formula (I) and forms thereof includes a compound wherein X is selected from carbonyl, amino-carbonyl or sulfonyl.

An example of a compound of formula (I) and forms thereof includes a compound wherein R₄ is selected from a C₁₋₄alkyl, aryl, aryl-C₁₋₄alkyl, heteroaryl, heteroaryl-C₁₋₄alkyl, heterocyclyl, C₃₋₁₂cycloalkyl or C₃₋₁₂cycloalkyl-C₁₋₄alkyl optionally substituted on aryl, heteroaryl or heterocyclyl with one, two or three substituents selected from C₁₋₆alkyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl or heterocyclyl.

An example of a compound of formula (I) and forms thereof includes a compound wherein R₄ is selected from a C₁₋₄alkyl, phenyl, naphthyl, phenyl-C₁₋₄alkyl, thienyl, furanyl, pyrazolyl, isoxazolyl, [1,2,3]thiadiazolyl, pyridinyl, pyrimidinyl, benzothienyl, indolyl, tetrazolyl-C₁₋₄alkyl, morpholinyl, piperidinyl, piperazinyl, benzo[1,3]dioxolyl, heterocyclyl-C₁₋₄alkyl, cyclopenyl, cyclohexyl, cycloheptyl or cyclohexyl-C₁₋₄alkyl optionally substituted on phenyl, thienyl, pyrazolyl, [1,2,3]thiadiazolyl, pyridinyl, indolyl, piperidinyl or C₃₋₁₂cycloalkyl with one, two or three substituents selected from C₁₋₆alkyl, C₁₋₆alkenyl, C₁₋₆alkynyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, phenyl, heteroaryl, morpholinyl or C₃₋₁₂cycloalkyl.

An example of a compound of formula (I) and forms thereof includes a compound wherein R₄ is selected from a C₁₋₄alkyl, phenyl, naphthyl, phenyl-C₁₋₄alkyl, thienyl, furanyl, pyrazolyl, isoxazolyl, [1,2,3]thiadiazolyl, pyridinyl, pyrimidinyl, benzothienyl, indolyl, tetrazolyl-C₁₋₄alkyl, morpholinyl, piperidinyl, piperazinyl, benzo[1,3]dioxolyl, cyclopenyl, cyclohexyl, cycloheptyl or cyclohexyl-C₁₋₄-alkyl optionally substituted on phenyl, thienyl, pyrazolyl, [1,2,3]thiadiazolyl, pyridinyl, indolyl, piperidinyl with one, two or three substituents selected from C₁₋₆alkyl, C₁₋₆alkoxy, C₁₋₆alkyl-carbonyl, amino, halogen, cyano, nitro, amino-sulfonyl, halo-C₁₋₄alkyl, phenyl or morpholinyl.

An example of a compound of formula (I) and forms thereof includes a compound wherein
R₁ is hydrogen or is selected from C₁₋₆alkyl, C₁₋₆alkoxy or amino-sulfonyl;
R₂ is hydrogen or is heterocyclyl optionally substituted with C₁₋₆alkyl;
R₃ is hydrogen or is C₁₋₄alkyl;
X is selected from carbonyl, amino-carbonyl, oxy-carbonyl or sulfonyl; and
R₄ is selected from a C₁₋₄alkyl, aryl, aryl-C₁₋₄alkyl, heteroaryl, heteroaryl-C₁₋₄alkyl, heterocyclyl, C₃₋₁₂cycloalkyl or C₃₋₁₂cycloalkyl-C₁₋₄alkyl optionally substituted on aryl, heteroaryl or heterocyclyl with one, two or three substituents selected from C₁₋₆alkyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl or heterocyclyl.

An example of a compound of formula (I) and forms thereof includes a compound wherein
R₁ is hydrogen or is selected from C₁₋₆alkyl, C₁₋₆alkoxy or amino-sulfonyl;
R₂ is hydrogen or is heterocyclyl optionally substituted with C₁₋₆alkyl;
R₃ is hydrogen or is C₁₋₄alkyl;
X is selected from carbonyl, amino-carbonyl, oxy-carbonyl or sulfonyl; and
R₄ is selected from a C₁₋₄alkyl, phenyl, naphthyl, phenyl-C₁₋₄alkyl, thienyl, furanyl, pyrazolyl, isoxazolyl, [1,2,3]thiadiazolyl, pyridinyl, pyrimidinyl, benzothienyl, indolyl, tetrazolyl-C₁₋₄alkyl, morpholinyl, piperidinyl, piperazinyl, benzo[1,3]dioxolyl, cyclopenyl, cyclohexyl, cycloheptyl or cyclohexyl-C₁₋₄alkyl optionally substituted on phenyl, thienyl, pyrazolyl, [1,2,3]thiadiazolyl, pyridinyl, indolyl, piperidinyl with one, two or three substituents selected from C₁₋₆alkyl, C₁₋₆alkoxy, C₁₋₆alkyl-carbonyl, amino, halogen, cyano, nitro, amino-sulfonyl, halo-C₁₋₄alkyl, phenyl or morpholinyl.

Examples of a compound of Formula (I) include compounds selected from the group consisting of:

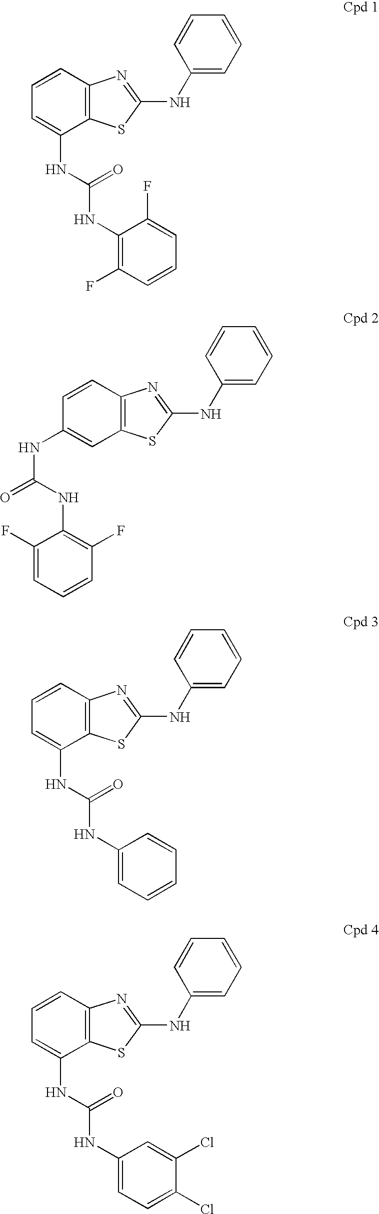

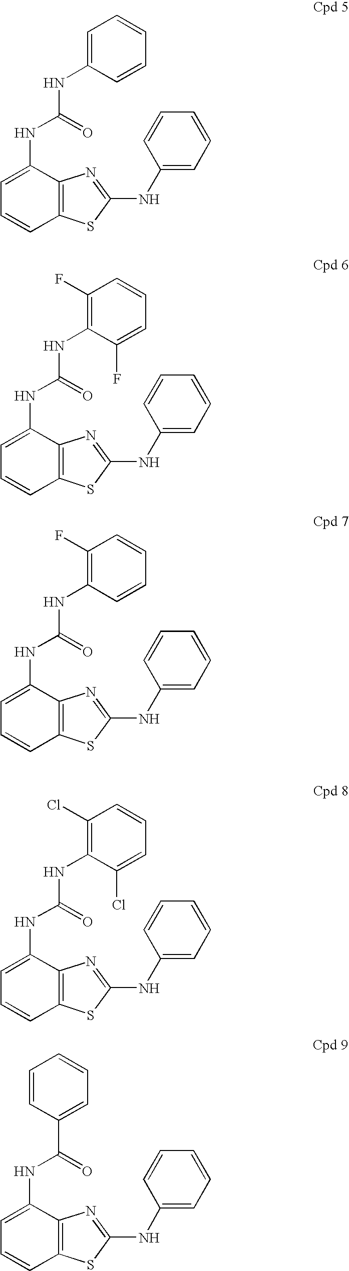

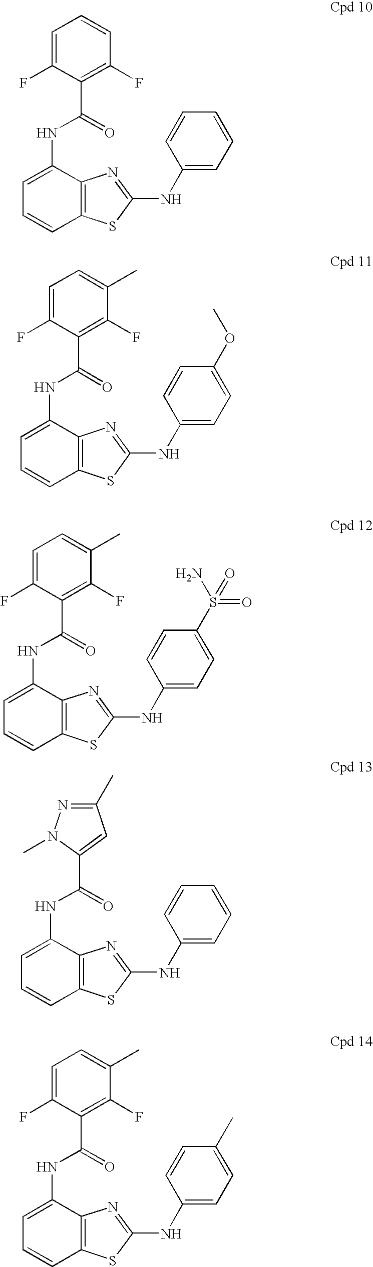

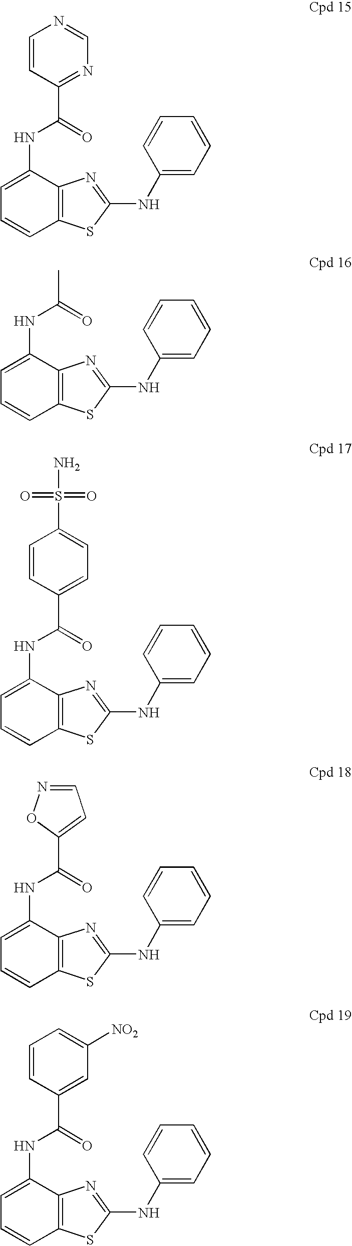

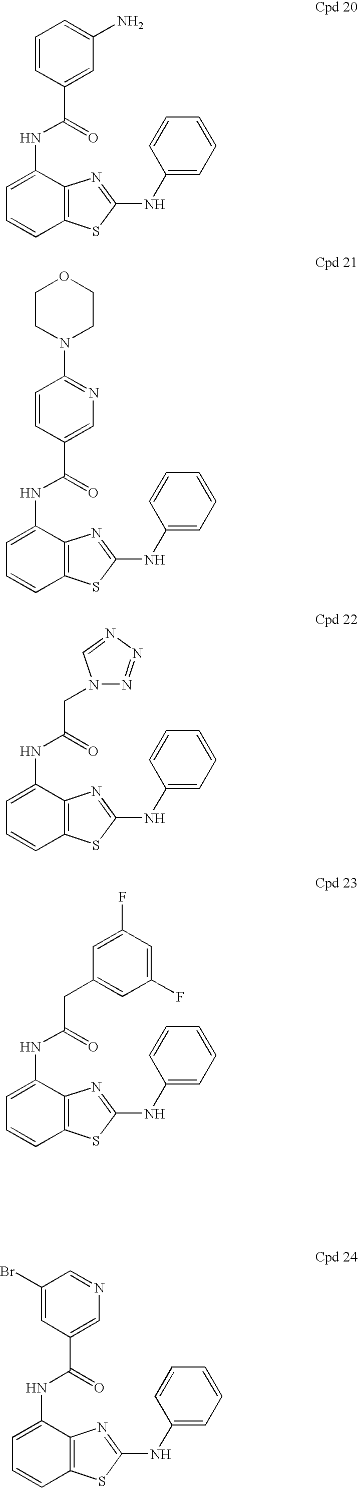

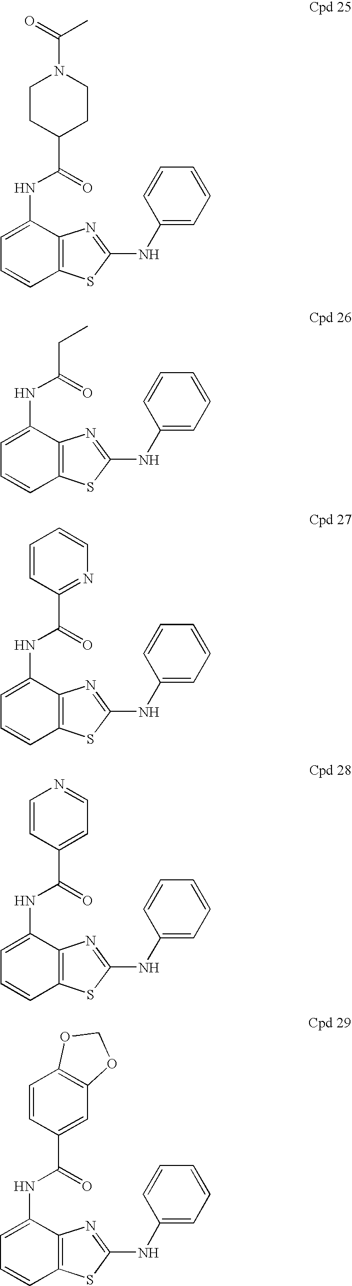

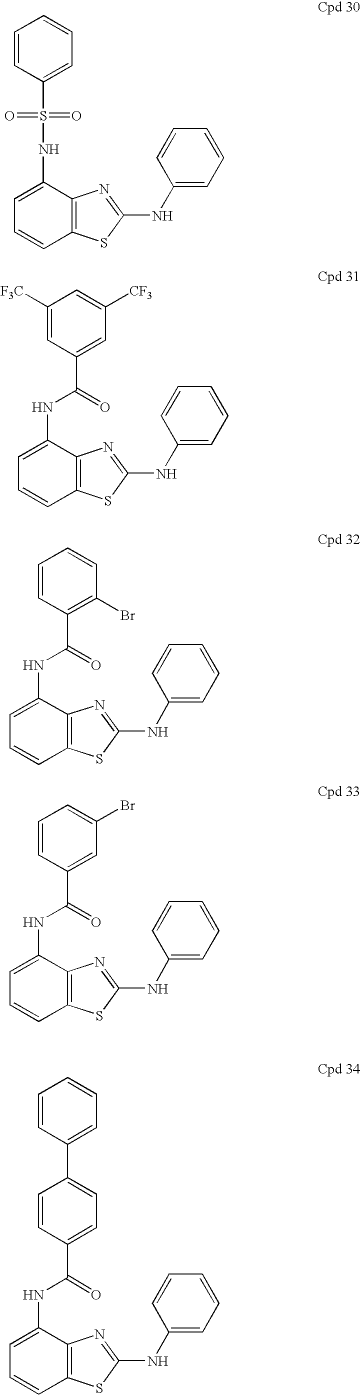

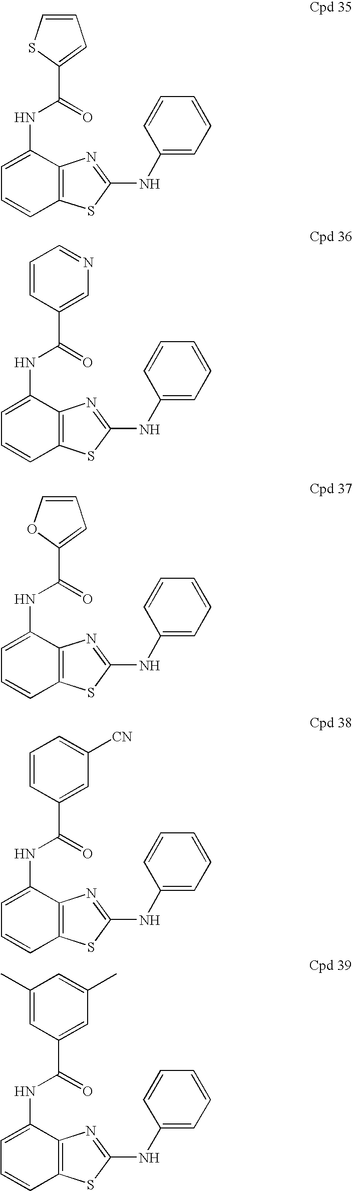

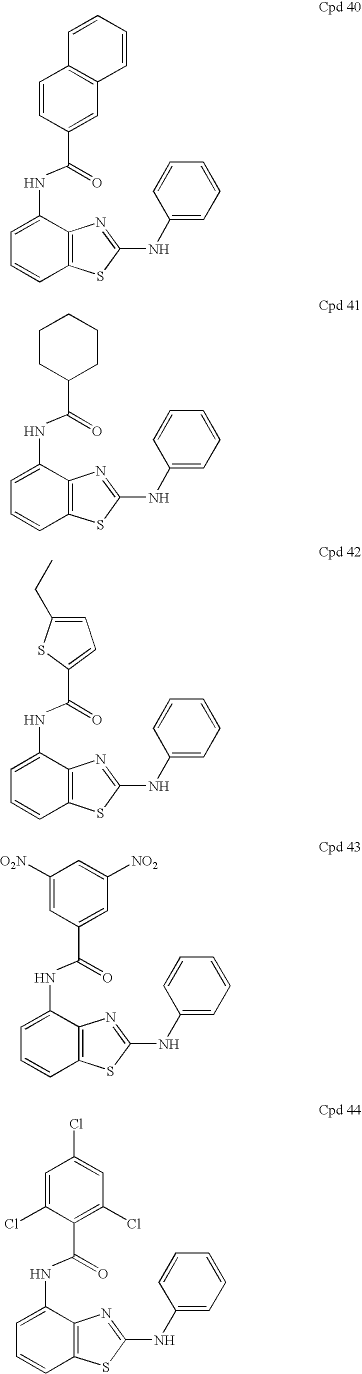

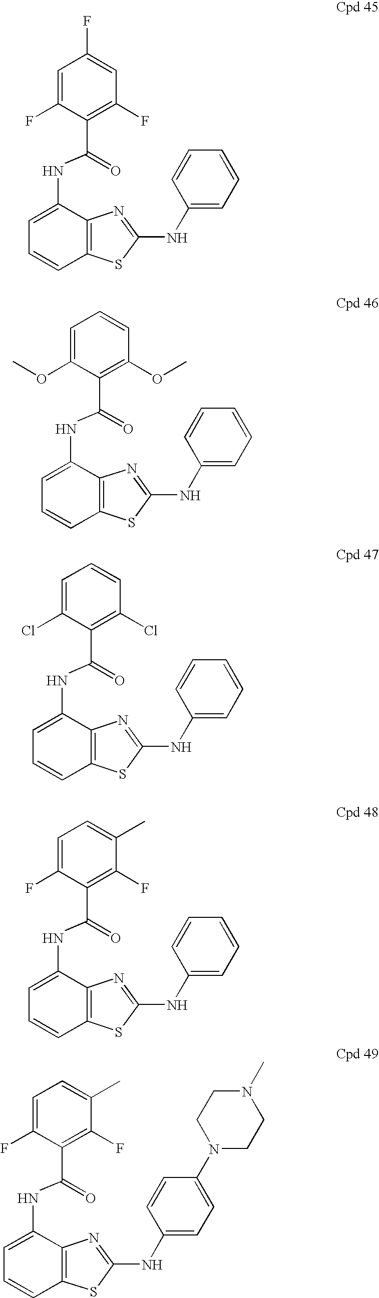

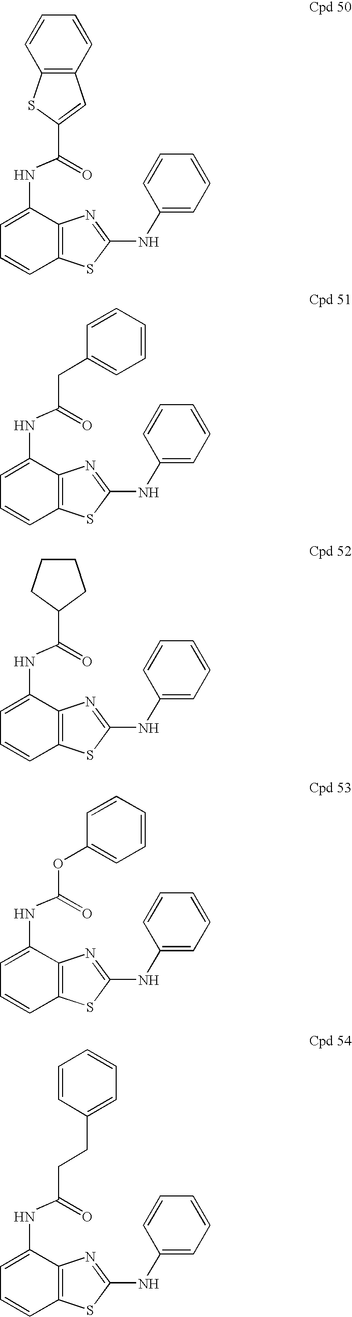

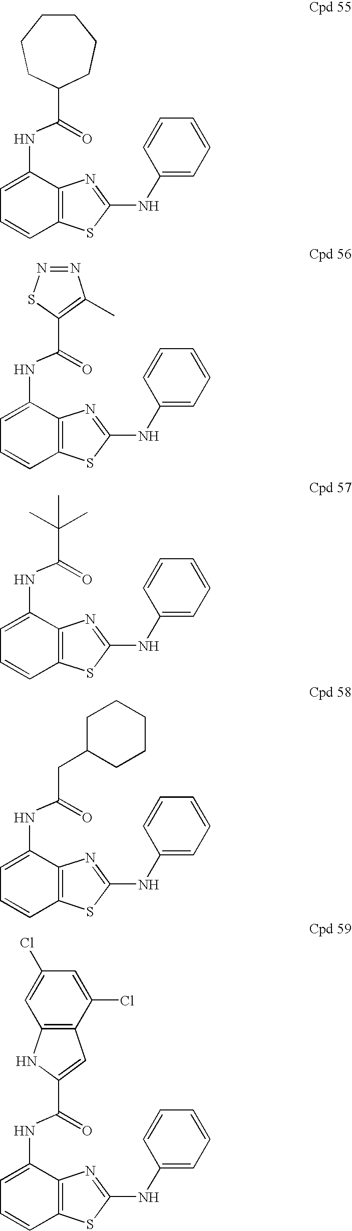

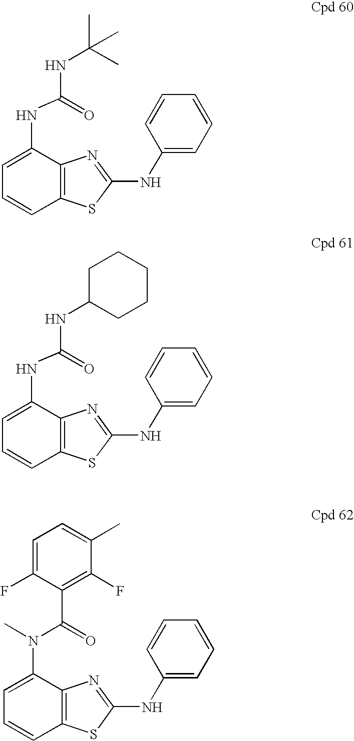

The present invention is further directed to certain compounds of Formula (Ia) which represent useful intermediates:

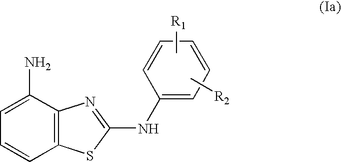

wherein
R₁ is hydrogen or is selected from C₁₋₆alkyl, C₁₋₆alkoxy, amino, halogen, cyano, amino-sulfonyl, C₁₋₄alkyl-amino-sulfonyl, halo-C₁₋₄alkyl or halo-C₁₋₄alkoxy; and,
R₂ is hydrogen or is selected from aryl, heteroaryl, heterocyclyl or C₃₋₁₂cycloalkyl optionally substituted with one or two substituents selected from C₁₋₆alkyl, C₁₋₆alkoxy or halogen.

An example of a compound of formula (I) and forms thereof includes a compound wherein R₁ is hydrogen or is selected from C₁₋₆alkyl, C₁₋₆alkoxy or amino-sulfonyl.

An example of a compound of formula (I) and forms thereof includes a compound wherein R₂ is hydrogen or is selected from heterocyclyl substituted with one C₁₋₆alkyl substituent.

Examples of the compound of Formula (Ia) include compounds selected from the group consisting of:

| Cpd | Name and Data |
|---|---|
| 2b | N²-phenyl-benzothiazole-2,4-diamine, |
| 5b | N²-(4-methoxy-phenyl)-benzothiazole-2,4-diamine, |
| 6b | 4-(4-amino-benzothiazol-2-ylamino)-benzenesulfonamide, and |
| 7b | N²-p-tolyl-benzothiazole-2,4-diamine. |

### Chemical Definitions & Nomenclature

Bond lines drawn into a ring system from a substituent variable indicate that the substituent may be attached to any of the substitutable ring atoms.

As used herein, the following terms are intended to have the following definitions. The definitions herein may specify that a chemical term has an indicated formula. The particular formula provided is not intended to limit the scope of the invention, but is provided as an illustration of the term. The scope of the per se definition of the term is intended to include the plurality of variations expected to be included by one of ordinary skill in the art.

The term “C₁₋₆alkyl” means a saturated aliphatic branched or straight-chain hydrocarbon radical or linking group having from 1 up to 6 carbon atoms in a linear or branched arrangement, wherein the radical is derived by the removal of one hydrogen atom from a carbon atom and the linking group is derived by the removal of one hydrogen atom from each of two carbon atoms in the chain. The term “C₁₋₆alkyl” also includes a “C₁₋₄alkyl” radical or linking group having from 1 up to 4 carbon atoms respectively, such as methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl and the like. Alkyl radicals may be attached to a core molecule and further substituted on any atom when allowed by available valences.

The term “C₂₋₆alkenyl” means an alkyl radical or linking group having from 2 up to 6 carbon atoms in a linear or branched arrangement having at least one carbon-carbon double bond. The term “C₂₋₆alkenyl” also includes a “C₂₋₄alkenyl” radical or linking group having from 2 up to 4 carbon atoms, such as ethenyl (also referred to as vinyl), iso-propenyl, allyl (also referred to as propenyl), propylidene and the like. Alkenyl radicals may be attached to a core molecule and further substituted on any atom when allowed by available valences.

The term “C₂₋₆alkynyl” means an alkyl radical or linking group having from 2 up to 6 carbon atoms in a linear or branched arrangement having at least one carbon-carbon triple bond. The term “C₂₋₆alkynyl” also includes a “C₂₋₄alkynyl” radical or linking group having from 2 up to 4 carbon atoms, such as ethynyl propynyl and the like. Alkynyl radicals may be attached to a core molecule and further substituted on any atom when allowed by available valences.

The term “C₁₋₆alkoxy” means an alkyl radical or linking group having from 1 up to 6 carbon atoms in a linear or branched arrangement, wherein the radical or linking group is attached through an oxygen linking atom, as in the formula: —O—C₁₋₆alkyl. The term “C₁₋₆alkoxy” also includes a “C₁₋₄alkoxy” radical or linking group having from 1 up to 6 carbon atoms and from 1 up to 4 carbon atoms respectively, such as methoxy, ethoxy, propoxy, butoxy and the like. An alkoxy radical may be attached to a core molecule and further substituted on any atom when allowed by available valences.

The term “C₃₋₁₂cycloalkyl” means a saturated or partially unsaturated cyclic hydrocarbon ring system radical. The term “C₃₋₁₂cycloalkyl” also includes a C₃₋₈cycloalkyl, C₃₋₁₀cycloalkyl, C₅₋₆cycloalkyl, C₅₋₈cycloalkyl, C₅₋₁₂cycloalkyl, C₉₋₁₂cycloalkyl or benzofused-C₃₋₁₂cycloalkyl ring system radical and the like, such as cyclopropyl, cyclobutyl, cyclopentyl, cyclohexyl, cycloheptyl, cyclooctyl, 1H-indenyl, indanyl, 9H-fluorenyl, 1,2,3,4-tetrahydro-naphthalenyl, acenaphthenyl, adamantanyl and the like. A C₃₋₁₂cycloalkyl radical may be attached to a core molecule and further substituted on any atom when allowed by available valences.

The term “benzofused-C₃₋₁₂cycloalkyl” means a C₃₋₁₂cycloalkyl ring system radical having a benzene ring fused on the ring system on adjacent carbons. A benzofused-C₃₋₁₂cycloalkyl radical may be attached to a core molecule and further substituted on any atom when allowed by available valences.

The term “aryl” means an unsaturated aromatic hydrocarbon ring system radical. Aryl ring systems include phenyl, naphthyl, azulenyl, anthracenyl and the like. An aryl radical may be attached to a core molecule and further substituted on any atom when allowed by available valences.

The term “hetero”, when used as a prefix for a ring system, refers to the replacement of at least one carbon atom member in the ring system with a heteroatom selected from N, O, S, S(O), or SO₂. A hetero ring may have 1, 2, 3 or 4 carbon atom members replaced by a nitrogen atom. Alternatively, a ring may have 1, 2 or 3 nitrogen atom members and 1 oxygen or sulfur atom member. Alternatively, a ring may have 1 oxygen or sulfur atom member. Alternatively, up to two adjacent ring members may be heteroatoms, wherein one heteroatom is nitrogen and the other heteroatom is selected from N, S or O.

The term “heterocyclyl” means a saturated or partially unsaturated “hetero” ring system radical. Heterocyclyl ring systems include azetidinyl, 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl and the like. The term “heterocyclyl” also includes a benzofused-heterocyclyl ring system radical and the like, such as indolinyl (also referred to as 2,3-dihydro-indolyl), benzo[1,3]dioxolyl, 2,3-dihydro-1,4-benzodioxinyl, 2,3-dihydro-benzofuranyl, 1,2-dihydro-phthalazinyl and the like. A heterocyclyl radical may be attached to a core molecule and further substituted on any atom when allowed by available valences.

The term “benzofused-heterocyclyl” means a heterocyclyl ring system radical having a benzene ring fused on the ring system on adjacent carbons. A benzofused-heterocyclyl radical may be attached to a core molecule and further substituted on any atom when allowed by available valences.

The term “heteroaryl” means a monovalent, unsaturated aromatic “hetero” ring system radical. Heteroaryl ring systems include furanyl, thienyl, pyrrolyl, oxazolyl, thiazolyl, imidazolyl, pyrazolyl, isoxazolyl, isothiazolyl, oxadiazolyl, triazolyl, thiadiazolyl, tetrazolyl, pyridinyl, pyridazinyl, pyrimidinyl, pyrazinyl and the like. The term “heteroaryl” also includes a benzofused-heteroaryl ring system radical and the like, such as indolizinyl, indolyl, azaindolyl, isoindolyl, benzofuranyl, benzothienyl, indazolyl, azaindazolyl, benzoimidazolyl, benzothiazolyl, benzoxazolyl, benzoisoxazolyl, benzothiadiazolyl, benzotriazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl and the like. A heteroaryl radical may be attached to a core molecule and further substituted on any atom when allowed by available valences.

The term “benzofused-heteroaryl” means a heteroaryl ring system radical having a benzene ring fused on the ring system on adjacent carbons. A benzofused-heteroaryl radical may be attached to a core molecule and further substituted on any atom when allowed by available valences.

The term “C₁₋₆alkoxy-carbonyl” means a radical of the formula: —C(O)—O—C₁₋₆alkyl, wherein C₁₋₆alkyl is optionally further substituted.

The term “C₁₋₄alkyl-amino-sulfonyl” means a radical of the formula: —SO₂—NH—C₁₋₄alkyl or —SO₂—N[C₁₋₄alkyl]₂, wherein C₁₋₄alkyl is optionally further substituted.

The term “C₁₋₆alkyl-carbonyl” means a radical of the formula: —C(O)—C₁₋₆alkyl, wherein C₁₋₆alkyl is optionally further substituted.

The term “amino” means a radical of the formula: —NH₂.

The term “amino-carbonyl” means a radical of the formula: —C(O)—NH₂, wherein NH₂ is optionally further substituted.

The term “amino-sulfonyl” means a radical of the formula: —SO₂—NH₂, wherein NH₂ is optionally further substituted.

The term “aryl-C₁₋₄alkyl” means a radical of the formula: —C₁₋₄alkyl-aryl.

The term “carbonyl” means a linking group of the formula: —C(O)—.

The term “C₃₋₁₂cycloalkyl-C₁₋₄alkyl” means a radical of the formula: —C₁₋₄alkyl-C₃₋₁₂cycloalkyl.

The term “halogen” or “halo” means the group chloro, bromo, fluoro or iodo.

The term “halo-C₁₋₄alkoxy” means a radical of the formula: —O—C₁₋₄alkyl-(halo)ₙ, wherein one or more halogen atoms may be substituted on C₁₋₄alkyl when allowed by available valences (wherein n represents that amount of available valences based on the number of carbon atoms in the chain), and includes monofluoromethoxy, difluoromethoxy, trifluoromethoxy, trifluoroethoxy and the like.

The term “halo-C₁₋₄alkyl” means a radical of the formula: —C₁₋₄alkyl-(halo), wherein one or more halogen atoms may be substituted on C₁₋₄alkyl when allowed by available valences (wherein n represents that amount of available valences based on the number of carbon atoms in the chain), and includes monofluoromethyl, difluoromethyl, trifluoromethyl, trifluoroethyl and the like.

The term “heteroaryl-C₁₋₄alkyl” means a radical of the formula: —C₁₋₄alkyl-heteroaryl.

The term “heterocyclyl-C₁₋₄alkyl” means a radical of the formula: —C₁₋₄alkyl-heterocyclyl.

The term “oxy-carbonyl” means a linking group of the formula: —C(O)—O—, wherein the —C(O)— portion is attached to the core molecule.

The term “sulfonyl” means a linking group of the formula: —SO₂—.

The term “substituted” means the independent replacement of one or more hydrogen atoms within a radical with that amount of substitutents allowed by available valences.

The term “dependently selected” means that the structure variables are specified in an indicated combination.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In general, IUPAC nomenclature rules are used herein.

### Compound Forms

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Suitable salt forms include acid addition salts which may, for example, be formed by mixing a solution of the compound according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulphuric acid, tartaric acid, trifluoroacetic acid and the like.

Furthermore when the compounds of the present invention carry an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts include the following: acetate, adipate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art. The scope of the present invention encompasses all such protected compound forms and mixtures thereof.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of the isolated species rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule which, in a given configuration, cannot be superimposed on its mirror image. This is in contrast to achiral molecules which can be superimposed on their mirror images.

The two distinct mirror image versions of the chiral molecule are also known as levo (left-handed), abbreviated L, or dextro (right-handed), abbreviated D, depending on which way they rotate polarized light. The symbols “R” and “S” represent the configuration of groups around a stereogenic carbon atom(s).

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levorotatory = \frac{( mass leverotatory )}{( mass dextrorotatory ) + ( mass levorotatory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( mass dextrorotatory )}{( mass dextrorotatory ) + ( mass levorotatory )} \times 100$$

The term “geometric isomer” means isomers that differ in the orientation of substituent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

Substituent atoms (other than hydrogen) attached to a ring system may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

The isomeric descriptors (“R,” “S,” “E,” and “Z”) indicate atom configurations and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the present invention may have one or more polymorph or amorphous crystalline forms and, as such, are intended to be included in the scope of the invention. In addition, some of the compounds may form solvates with water (i.e., hydrates) or common organic solvents (e.g., organic esters such as ethanolate and the like) and, as such, are also intended to be encompassed within the scope of this invention.

### Methods of Use

The compounds of formula (I) are inhibitors of a protein kinase such as CDK or VEGF, having an IC₅₀ (50% inhibition concentration) or an EC₅₀ (50% effective concentration) in a range of about 50 μM or less, of about 25 μM or less, of about 15 μM or less, of about 10 μM or less, of about 5 μM or less, of about 1 μM or less, of about 0.5 μM or less, of about 0.25 μM or less or of about 0.1 μM or less.

The present invention includes a compound of formula (I) and forms thereof as a protein kinase inhibitor, wherein the CDK protein kinase is CDK-1 or CDK-2 and the VEGF protein kinase is VEGF-R2.

The present invention includes a prodrug form of a compound of formula (I) and forms thereof as a protein kinase inhibitor.

The present invention includes a metabolite form of a compound of formula (I) and forms thereof as a protein kinase inhibitor.

The present invention includes an isolated form of a compound of formula (I) and forms thereof as a protein kinase inhibitor.

The present invention includes a compound of formula (I) or a form thereof,

wherein the compound is labeled with a ligand for use as a marker, and wherein the ligand is a radioligand selected from deuterium, tritium and the like.

The present invention includes use of a compound of formula (I) and forms thereof as an inhibitor of a protein kinase such as CDK-1, CDK-2 or VEGF-R2 comprising contacting the protein kinase domain or receptor with the compound.

The present invention includes the use of a compound of formula (I) and forms thereof as a pharmaceutical composition, medicine or medicament for treating a kinase mediated disease, disorder or condition.

The present invention includes the use of a compound of formula (I) and forms thereof as a medicament.

The present invention includes the use of a prodrug of a compound of formula (I) and forms thereof as a pharmaceutical composition, medicine or medicament for treating a kinase mediated disease, disorder or condition.

The present invention includes the use of a prodrug of a compound of formula (I) and forms thereof as a medicament.

The present invention is directed to a method for treating a chronic or acute protein kinase mediated disease, disorder or condition in a subject in need thereof comprising administering to the subject an effective amount of a compound of formula (I) and forms thereof.

The method of the present invention further comprises administering to the subject an effective amount of a prodrug of a compound of formula (I) and forms thereof.

The method of the present invention further comprises treating a chronic or acute CDK-1, CDK-2 or VEGF-R2 mediated disease, disorder or condition.

The method of the present invention wherein the disease, disorder or condition is associated with increased or unregulated protein kinase activity, expression or signaling and the like in the subject.

The method of the present invention further comprises administering to the subject an effective amount of a compound of formula (I) as a pharmaceutical composition, medicine or medicament thereof.

The term “chronic or acute protein kinase mediated disease, disorder or condition” as used herein, includes, and is not limited to diseases, disorders or conditions associated with unregulated kinase activity and conditions that accompany such activity.

The term “unregulated protein kinase activity, expression or signaling” refers to 1) increased or unregulated kinase expression or signaling, 2) increased kinase expression leading to unregulated cell proliferation, 3) increased kinase signalling leading to unregulated cell proliferation, or 4) mutations leading to constitutive kinase activation. The existence of unregulated kinase activity may be determined by procedures well known in the art.

The term “unregulated cell proliferation” refers to cell proliferation of one or more subset of cells in a multicellular organism resulting in harm (such as discomfort or decreased life expectancy) to the multicellular organism.

Tumor cells which result from unregulated cell proliferation use many mechanisms to enhance their survival and spread and often have high rates of proliferation because growth control signals that keep normal cells in check are defective. Many tumor cells secrete autocrine growth factors that increase proliferation rates or they induce other cells to secrete growth factors that they utilize.

Tumor cells grow and spread by dislodging from a primary tumor site, using proteases to digest the extracellular matrix, spreading in response to migration cues, allowing them to migrate to certain tissues preferentially where overexpressed adhesion molecules allow attachment and growth at the new site. The totality of these and other biological processes are responsible for the lethal effects of a tumor. A kinase inhibitor may affect one or more aspects of tumor survival mechanisms and thus be therapeutically useful. Alternatively, a kinase inhibitor may not affect one particular tumor survival mechanism but may still be therapeutically useful by affecting tumor survival by an unknown or as yet unelucidated mechanism of action.

The foregoing methods contemplate that a compound of formula (I) or a form thereof is useful for treating diseases, disorders or conditions such as, without limitation, osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohns disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, occular diseases, viral infections, heart disease, lung or pulmonary diseases or kidney or renal diseases.

Certain diseases, disorders or conditions further include, without limitation, acute or chronic cancer selected from bladder cancer, brain, head or neck cancer, breast cancer, colorectal cancer, endometrial cancer, epidermoid cancer, esophageal cancer, gastric cancer, glioma cancer, lung cancer, ovarian cancer, pancreatic cancer, prostate cancer, renal cell cancer, Kaposi's sarcoma, leukemia, lymphoma or papillocarcinoma; and, cancer-associated pathologies selected from abnormal cell proliferation, unregulated cell proliferation, tumor growth, tumor angiopathy, tumor angiogenesis, tumor vascularization or metastatic cancer cell invasion and migration.

Certain diseases, disorders or conditions further include, without limitation, fibroproliferative and differentiative skin diseases or disorders selected from papilloma formation, psoriasis, dermatitis, eczema, seborrhea or chemotherapy-induced alopecia; central nervous system diseases selected from Alzheimer's disease, Parkinson's disease or depression; occular diseases selected from macular degeneration, diseases of the cornea or glaucoma; viral infections selected from mycotic infection, autoimmune disease or cytomegalovirus; heart disease selected from atherosclerosis, neointima formation or transplantation-induced vasculopathies such as arterial restenosis; lung or pulmonary diseases selected from allergic-asthma, lung fibrosis, pulmonary fibrosis or chronic obstructive pulmonary disorder; and, kidney or renal diseases selected from acute, subacute or chronic forms of glomerulonephritis or membranoproliferative glomerulonephritis, glomerulosclerosis, congenital multicystic renal dysplasia or kidney fibrosis.

The term “administering,” with respect to the methods of the present invention, refers to a means for treating a disease, disorder or syndrome as described herein with a compound of formula (I) or a form thereof, which would obviously be included within the scope of the invention albeit not specifically disclosed for certain of said compounds.

Such methods include therapeutically or prophylactically administering an effective amount of compound of formula (I) or a form thereof at different times during the course of a therapy or concurrently in a combination form. Such methods further include administering an effective amount of said compound with one or more agents at different times during the course of a therapy or concurrently in a combination form.

The term “prodrug” means a compound of formula (I) or a form thereof that is converted in vivo into a functional derivative form that may contribute to therapeutic biological activity, wherein the converted form may be: 1) a relatively active form; 2) a relatively inactive form; 3) a relatively less active form; or, 4) any form which results, directly or indirectly, from such in vivo conversions.

Prodrugs are useful when said compound may be either too toxic to administer systemically, absorbed poorly by the digestive tract or broken down by the body before it reaches its target. Conventional procedures for the selection and preparation of suitable prodrug derivatives are described in, for example, “Design of Prodrugs”, ed. H. Bundgaard, Elsevier, 1985.

The term “metabolite” means a form of a compound of formula (I) or a form thereof converted by in vivo metabolism or a metabolic process to a derivative of said compound.

The term “subject” as used herein, refers to a patient, such as an animal, a mammal or a human, who has been the object of treatment, observation or experiment and is at risk of (or susceptible to) developing a disease or disorder or having a disease or disorder related to unregulated kinase activity.

The term “effective amount” refers to that amount of a compound of formula (I) or a form, pharmaceutical composition, medicine or medicament thereof that elicits the biological or medicinal response (such as inhibiting activation of unregulated kinase activity) in a tissue system, animal or human, that is being sought by a researcher, veterinarian, medical doctor, or other clinician, which includes alleviation of the symptoms of the disease or disorder being treated.

The effective amount of said compound is from about 0.001 mg/kg/day to about 300 mg/kg/day.

The term “pharmaceutical composition” refers to a product containing a compound of formula (I) or a form thereof, such as a product comprising the specified ingredients in the specified amounts, as well as any product which results, directly or indirectly, from such combinations of the specified ingredients in the specified amounts.

The term “medicament” or “medicine” refers to a product containing a compound of formula (I) or a form thereof. The present invention includes use of such a medicament for treating a chronic or acute kinase mediated disease, disorder or condition.

The term “pharmaceutically acceptable” refers to molecular entities and compositions that are of sufficient purity and quality for use in the formulation of a pharmaceutical composition, medicine or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a pharmaceutical composition, medicine or medicament for either human or veterinary use.

The term “combination form” refers to the use of a combination product comprising a compound of formula (I) or a form, pharmaceutical composition, medicine or medicament thereof and at least one therapeutic agent for treating a chronic or acute protein kinase mediated disease, disorder or condition.

Advantageously, the effective amount of a combination product for treating a chronic or acute protein kinase mediated disease, disorder or condition may be a reduced amount of either or both the compound or therapeutic agent compared to the effective amount of the compound or therapeutic agent otherwise recommended for treating the disease, disorder or condition. Therefore, it is contemplated that the compound is administered to the subject before, during or after the time the agent is administered.

The term “therapeutic agent” refers to chemotherapeutic agents used to treat a kinase mediated cancer or antiviral agents used to treat cytomegalovirus. Chemotherapeutic agents include and are not limited to anti-angiogenic agents, anti-tumor agents, cytotoxic agents, inhibitors of cell proliferation, radiation therapy and the like or a combination thereof.

The term “treating” refers, without limitation, to facilitating the eradication of, inhibiting the progression of or promoting stasis of a chronic or acute kinase mediated disease, disorder or condition.

The term “radiation therapy” refers to a therapy that comprises exposing the subject in need thereof to radiation. The present invention includes a method for administering a compound of formula (I) or a form, pharmaceutical composition, medicine or medicament thereof in combination with radiation therapy. Procedures for administering such therapy are known to those skilled in the art. The appropriate scheme of radiation therapy will be similar to those already employed in clinical therapies wherein the radiation therapy is used alone or in combination with other chemotherapeutic agents.

The present invention includes a pharmaceutical composition comprising an admixture of a compound of formula (I) or a form thereof and one or more pharmaceutically acceptable excipients.

The present invention includes a process for making a pharmaceutical composition, medicine or medicament comprising mixing a compound of formula (I) or a form thereof and an optional pharmaceutically acceptable carrier. The present invention includes a pharmaceutical composition, medicine or medicament resulting from the process of mixing a compound of formula (I) or a form thereof and an optional pharmaceutically acceptable carrier. Contemplated processes include both conventional and unconventional pharmaceutical techniques.

Said pharmaceutical composition, medicine or medicament may take a wide variety of forms to effectuate mode of administration, wherein the mode includes, and is not limited to, intravenous (both bolus and infusion), oral, nasal, transdermal, topical with or without occlusion, and via injection intraperitoneally, subcutaneously, intramuscularly, intratumorally, intracerebrally or intracranially. The composition, medicine or medicament may be in a dosage unit such as a tablet, pill, capsule, powder, granule, sterile parenteral solution or suspension, metered aerosol or liquid spray, drop, ampoule, auto-injector device or suppository for such administration modes.

Pharmaceutical compositions, medicines or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions. Alternatively, the pharmaceutical composition, medicine or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the pharmaceutical composition, medicine or medicament contains an effective amount of the active ingredient necessary to be therapeutically or prophylactically effective as described above. The pharmaceutical composition, medicine or medicament may contain from about 0.001 mg to about 5000 mg (preferably, from about 0.001 to about 500 mg) of a compound of formula (I) or a form thereof and may be constituted into any form suitable for the mode of administration selected for a subject in need.

An example of a contemplated effective amount for a pharmaceutical composition, medicine or medicament of the present invention may range from about 0.001 mg to about 300 mg/kg of body weight per day. In another example, the range is from about 0.003 to about 100 mg/kg of body weight per day. In another example, the range is from about 0.005 to about 15 mg/kg of body weight per day. The pharmaceutical composition, medicine or medicament may be administered according to a dosage regimen of from about 1 to about 5 times per day.

For oral administration, the pharmaceutical composition, medicine or medicament is preferably in the form of a tablet containing, e.g., 0.01, 0.05, 0.1, 0.5, 1.0, 2.5, 5.0, 10.0, 15.0, 25.0, 50.0, 100, 150, 200, 250 and 500 milligrams of a compound of formula (I) or a form thereof for the symptomatic adjustment of the dosage to the patient to be treated. Optimal dosages will vary depending on factors associated with the particular patient being treated (e.g., age, weight, diet and time of administration), the severity of the condition being treated, the particular compound being used, the mode of administration and the strength of the preparation. The use of either daily administration or post-periodic dosing may be employed.

A representative compound of formula (I) or a form thereof includes a compound selected from the group consisting of:

| Cpd | Name |
|---|---|
| 1 | 1-(2,6-dichloro-phenyl)-3-(2-phenylamino-benzothiazol-4-yl)-urea, |
| 2 | 1-(2,6-difluoro-phenyl)-3-(2-phenylamino-benzothiazol-6-yl)-urea, |
| 3 | 1-phenyl-3-(2-phenylamino-benzothiazol-7-yl)-urea, |
| 4 | 1-(3,4-dichloro-phenyl)-3-(2-phenylamino-benzothiazol-7-yl)-urea, |
| 5 | 1-phenyl-3-(2-phenylamino-benzothiazol-4-yl)-urea, |
| 6 | 1-(2,6-difluoro-phenyl)-3-(2-phenylamino-benzothiazol-4-yl)-urea, |
| 7 | 1-(2-fluoro-phenyl)-3-(2-phenylamino-benzothiazol-4-yl)-urea, |
| 8 | 1-(2,6-dichloro-phenyl)-3-(2-phenylamino-benzothiazol-4-yl)-urea, |
| 9 | N-(2-phenylamino-benzothiazol-4-yl)-benzamide, |
| 10 | 2,6-difluoro-N-(2-phenylamino-benzothiazol-4-yl)-benzamide, |
| 11 | 2,6-difluoro-N-[2-(4-methoxy-phenylamino)-benzothiazol-4-yl]-3-methyl- |
| | benzamide, |
| 12 | 2,6-difluoro-3-methyl-N-[2-(4-sulfamoyl-phenylamino)-benzothiazol-4-yl]- |
| | benzamide, |
| 13 | 2,5-dimethyl-2H-pyrazole-3-carboxylic acid (2-phenylamino-benzothiazol-4- |
| | yl)-amide, |
| 14 | 2,6-difluoro-3-methyl-N-(2-p-tolylamino-benzothiazol-4-yl)-benzamide, |
| 15 | pyrimidine-4-carboxylic acid (2-phenylamino-benzothiazol-4-yl)-amide, |
| 16 | N-(2-phenylamino-benzothiazol-4-yl)-acetamide, |
| 17 | N-(2-phenylamino-benzothiazol-4-yl)-4-sulfamoyl-benzamide, |
| 18 | isoxazole-5-carboxylic acid (2-phenylamino-benzothiazol-4-yl)-amide, |
| 19 | 3-nitro-N-(2-phenylamino-benzothiazol-4-yl)-benzamide, |
| 20 | 3-amino-N-(2-phenylamino-benzothiazol-4-yl)-benzamide, |
| 21 | 6-morpholin-4-yl-N-(2-phenylamino-benzothiazol-4-yl)-nicotinamide, |
| 22 | N-(2-phenylamino-benzothiazol-4-yl)-2-tetrazol-1-yl-acetamide, |
| 23 | 2-(3,5-difluoro-phenyl)-N-(2-phenylamino-benzothiazol-4-yl)-acetamide, |
| 24 | 5-bromo-N-(2-phenylamino-benzothiazol-4-yl)-nicotinamide, |
| 25 | 1-acetyl-piperidine-4-carboxylic acid (2-phenylamino-benzothiazol-4-yl)- |
| | amide, |
| 26 | N-(2-phenylamino-benzothiazol-4-yl)-propionamide, |
| 27 | pyridine-2-carboxylic acid (2-phenylamino-benzothiazol-4-yl)-amide, |
| 28 | N-(2-phenylamino-benzothiazol-4-yl)-isonicotinamide, |
| 29 | benzo[1,3]dioxole-5-carboxylic acid (2-phenylamino-benzothiazol-4-yl)- |
| | amide, |
| 30 | N-(2-phenylamino-benzothiazol-4-yl)-benzenesulfonamide, |
| 31 | N-(2-phenylamino-benzothiazol-4-yl)-3,5-bis-trifluoromethyl-benzamide, |
| 32 | 2-bromo-N-(2-phenylamino-benzothiazol-4-yl)-benzamide, |
| 33 | 3-bromo-N-(2-phenylamino-benzothiazol-4-yl)-benzamide, |
| 34 | biphenyl-4-carboxylic acid (2-phenylamino-benzothiazol-4-yl)-amide, |
| 35 | thiophene-2-carboxylic acid (2-phenylamino-benzothiazol-4-yl)-amide, |
| 36 | N-(2-phenylamino-benzothiazol-4-yl)-nicotinamide, |
| 37 | furan-2-carboxylic acid (2-phenylamino-benzothiazol-4-yl)-amide, |
| 38 | 3-cyano-N-(2-phenylamino-benzothiazol-4-yl)-benzamide, |
| 39 | 3,5-dimethyl-N-(2-phenylamino-benzothiazol-4-yl)-benzamide, |
| 40 | naphthalene-2-carboxylic acid (2-phenylamino-benzothiazol-4-yl)-amide, |
| 41 | cyclohexanecarboxylic acid (2-phenylamino-benzothiazol-4-yl)-amide, |
| 42 | 5-ethyl-thiophene-2-carboxylic acid (2-phenylamino-benzothiazol-4-yl)-amide, |
| 43 | 3,5-dinitro-N-(2-phenylamino-benzothiazol-4-yl)-benzamide, |
| 44 | 2,4,6-trichloro-N-(2-phenylamino-benzothiazol-4-yl)-benzamide, |
| 45 | 2,4,6-trifluoro-N-(2-phenylamino-benzothiazol-4-yl)-benzamide, |
| 46 | 2,6-dimethoxy-N-(2-phenylamino-benzothiazol-4-yl)-benzamide, |
| 47 | 2,6-dichloro-N-(2-phenylamino-benzothiazol-4-yl)-benzamide, |
| 48 | 2,6-difluoro-3-methyl-N-(2-phenylamino-benzothiazol-4-yl)-benzamide, |
| 49 | 2,6-difluoro-3-methyl-N-{2-[4-(4-methyl-piperazin-1-yl)-phenylamino]- |
| | benzothiazol-4-yl}-benzamide, |
| 50 | benzo[b]thiophene-2-carboxylic acid (2-phenylamino-benzothiazol-4-yl)- |
| | amide, |
| 51 | 2-phenyl-N-(2-phenylamino-benzothiazol-4-yl)-acetamide, |
| 52 | cyclopentanecarboxylic acid (2-phenylamino-benzothiazol-4-yl)-amide, |
| 53 | (2-phenylamino-benzothiazol-4-yl)-carbamic acid phenyl ester, |
| 54 | 3-phenyl-N-(2-phenylamino-benzothiazol-4-yl)-propionamide, |
| 55 | cycloheptanecarboxylic acid (2-phenylamino-benzothiazol-4-yl)-amide, |
| 56 | 4-methyl-[1,2,3]thiadiazole-5-carboxylic acid (2-phenylamino-benzothiazol-4- |
| | yl)-amide, |
| 57 | 2,2-dimethyl-N-(2-phenylamino-benzothiazol-4-yl)-propionamide, |
| 58 | 2-cyclohexyl-N-(2-phenylamino-benzothiazol-4-yl)-acetamide, |
| 59 | 4,6-dichloro-1H-indole-2-carboxylic acid (2-phenylamino-benzothiazol-4-yl)-amide, |
| 60 | 1-tert-butyl-3-(2-phenylamino-benzothiazol-4-yl)-urea, |
| 61 | 1-cyclohexyl-3-(2-phenylamino-benzothiazol-4-yl)-urea, or |
| 62 | 2,6-difluoro-3,N-dimethyl-N-(2-phenylamino-benzothiazol-4-yl)-benzamide. |

A representative compound of formula (I) or a form thereof includes a compound selected from the group consisting of:

| Cpd | Name |
|---|---|
| 1 | 1-(2,6-dichloro-phenyl)-3-(2-phenylamino-benzothiazol-4-yl)-urea, |
| 10 | 2,6-difluoro-N-(2-phenylamino-benzothiazol-4-yl)-benzamide, |
| 13 | 2,5-dimethyl-2H-pyrazole-3-carboxylic acid (2-phenylamino- |
| | benzothiazol-4-yl)-amide, |
| 14 | 2,6-difluoro-3-methyl-N-(2-p-tolylamino-benzothiazol-4-yl)- |
| | benzamide, |
| 17 | N-(2-phenylamino-benzothiazol-4-yl)-4-sulfamoyl-benzamide, |
| 21 | 6-morpholin-4-yl-N-(2-phenylamino-benzothiazol-4-yl)- |
| | nicotinamide, |
| 22 | N-(2-phenylamino-benzothiazol-4-yl)-2-tetrazol-1-yl-acetamide, |
| 23 | 2-(3,5-difluoro-phenyl)-N-(2-phenylamino-benzothiazol-4-yl)- |
| | acetamide, |
| 26 | N-(2-phenylamino-benzothiazol-4-yl)-propionamide, |
| 28 | N-(2-phenylamino-benzothiazol-4-yl)-isonicotinamide, |
| 30 | N-(2-phenylamino-benzothiazol-4-yl)-benzenesulfonamide, |
| 31 | N-(2-phenylamino-benzothiazol-4-yl)-3,5-bis-trifluoromethyl- |
| | benzamide, |
| 36 | N-(2-phenylamino-benzothiazol-4-yl)-nicotinamide, |
| 39 | 3,5-dimethyl-N-(2-phenylamino-benzothiazol-4-yl)-benzamide, |
| 41 | cyclohexanecarboxylic acid (2-phenylamino-benzothiazol- |
| | 4-yl)-amide, |
| 44 | 2,4,6-trichloro-N-(2-phenylamino-benzothiazol-4-yl)-benzamide, |
| 45 | 2,4,6-trifluoro-N-(2-phenylamino-benzothiazol-4-yl)-benzamide, |
| 47 | 2,6-dichloro-N-(2-phenylamino-benzothiazol-4-yl)-benzamide, |
| 48 | 2,6-difluoro-3-methyl-N-(2-phenylamino-benzothiazol-4-yl)- |
| | benzamide, |
| 49 | 2,6-difluoro-3-methyl-N-{2-[4-(4-methyl-piperazin-1-yl)- |
| | phenylamino]- |
| | benzothiazol-4-yl}-benzamide, |
| 51 | 2-phenyl-N-(2-phenylamino-benzothiazol-4-yl)-acetamide, or |
| 52 | cyclopentanecarboxylic acid (2-phenylamino-benzothiazol- |
| | 4-yl)-amide. |

### Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

General: ¹H and ¹³C NMR spectra were measured on a Bruker AC-300 (300 MHz) spectrometer using tetramethylsilane and the deuterated solvent respectively as internal standards. Elemental analyses were obtained by Quantitative Technologies Inc. (Whitehouse, N.J.) and the results were within 0.4% of the calculated values unless otherwise mentioned. Melting points were determined in open capillary tubes with a MeI-Temp II apparatus (Laboratory Devices Inc.) and were uncorrected. Electrospray mass spectra (MS-ES) were recorded on a Hewlett Packard 59987A spectrometer. High resolution mass spectra (HRMS) were obtained on a Micromass Autospec. E spectrometer by fast atom bombardment (FAB) technique.

The terms used in describing the invention are commonly used and known to those skilled in the art. As used herein, the following abbreviations and formulas have the indicated meanings:

Cpd compound
DCM dichloromethane
DIC diisopropyl carbodiimide

### DMF N,N-dimethylformamide

DMSO dimethyl sulfoxide
HOBt 1-hydroxybenzotriazole
IPA isopropanol
min minute(s)
h/hr/hrs hour(s)
psi pounds per square inch
Et₃N or TEA triethylamine
THF tetrahydrofuran

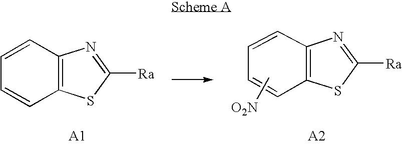

Compound A1 (wherein Ra is a halogen leaving group) is reacted with a strong acid (such as concentrated H₂SO₄, concentrated HNO₃ and the like and mixtures thereof) to provide a Compound A2.

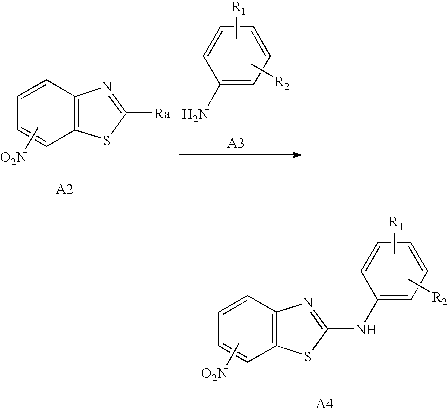

A solution of Compound A2 (1 equivalent) (in a solvent such as THF, IPA and the like and mixtures thereof) is reacted with a Compound A3 (20 equivalents), in the presence of a reagent (2 equivalents) (such as K₂CO₃ and the like) to generate a Compound A4.

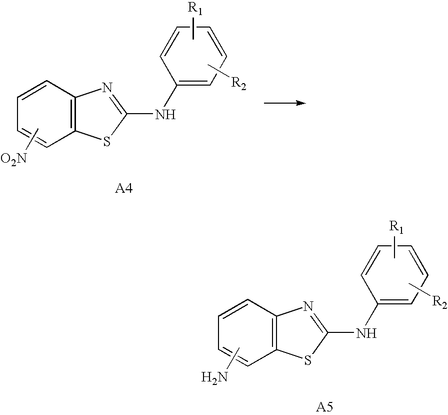

Compound A4 was reacted with a reducing metal (such as iron powder and the like) in the presence of an acid (such as HCl, acetic acid and the like) or by hydrogenation (using hydrogen gas under pressure in the range of from about 30 to about 50 psi) in the presence of a catalyst (such as Raney nickel, palladium on carbon and the like) to generate a Compound A5.

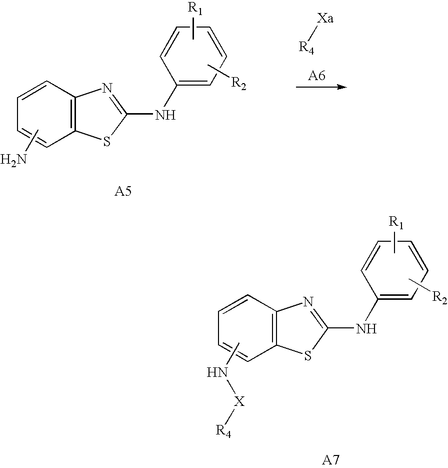

A solution of Compound A5 (1 equivalent) (in a solvent such as CH₂Cl₂, DMF and the like) is reacted with a Compound A6 (1 equivalent) (wherein Xa is a reactive group such as isocyanato, acid chloride, carboxylic acid and the like and wherein certain portions of Xa are incorporated into X as a product of the reaction) in the optional presence of a reagent to provide a Compound A7, representative of a compound of formula (I).

Certain functional group transformations (using a reactive group such as an acid chloride and the like) require the presence of 2 equivalents of a reagent (such as TEA and the like); others (using a reactive group such as carboxylic acid and the like) require the presence of 1 equivalent of a reagent (such as HOBt, DIC and the like and mixtures thereof). One skilled in the art would understand which reaction conditions are optimum for particular functional group transformations.

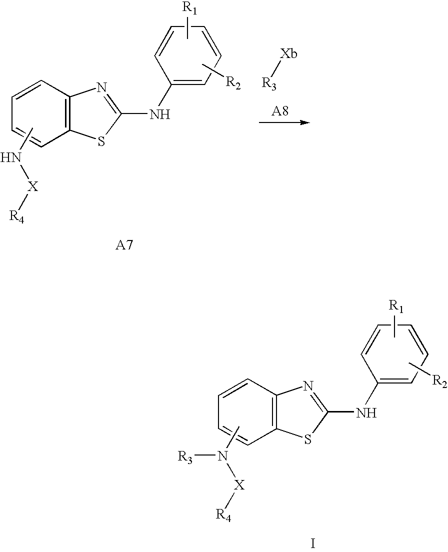

A solution of Compound A7 (in a solvent such as DMF and the like), in the presence of a reagent (such as NaH and the like), is reacted with a Compound A8 (wherein Xb is a halogen leaving group) to generate a compound of formula (I).

### EXAMPLE 1

### 1-(2,6-difluoro-phenyl)-3-(2-phenylamino-benzothiazol-7-yl)-urea Compound 1

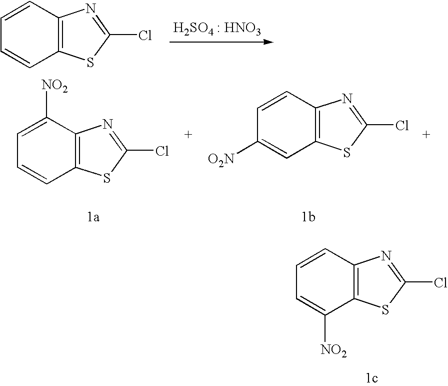

To a flask was added conc. H₂SO₄ (30 mL) and conc. HNO₃ (15 mL) slowly at 0° C., then 2-chloro-benzothiazole (5.0 g, 29.5 mmol) was added dropwise. After stirring at 0° C. for 1 hr, the mixture was poured into ice slowly. After being warmed to room temperature, the solid was collected by filtration, washed with water, air dried, then purified by flash chromatography (silica gel, DCM:hexane/5:5) to afford 0.51 g (8%) of 2-chloro-4-nitro-benzothiazole Compound 1a, 3.92 g (62%) of 2-chloro-6-nitro-benzothiazole Compound 1b and 1.46 g (23%) of 2-chloro-7-nitro-benzothiazole

Compound 1c. Alternatively, Compound 1a, Compound 1b and Compound 1c are commercially available.

Compound 1a: ¹H NMR (400 MHz, CDCl₃) δ 8.20 (d, J=7.9 Hz, 1H), 8.04 (d, J=8.0, 1H), 7.57 (t, J=7.9 Hz, 1H). MS (ESI) m/z: 215 (M+H)⁺.

Compound 1b: ¹H NMR (400 MHz, CDCl₃) δ 8.78 (s, 1H), 8.35 (d, J=8.3 Hz, 1H), 8.05 (d, J=8.4 Hz, 1H). MS (ESI) m/z: 215 (M+H)⁺.

Compound 1c: ¹H NMR (400 MHz, CDCl₃) δ 8.42 (d, J=7.9 Hz, 1H), 8.29 (d, J=8.0 Hz, 1H), 7.70 (t, J=8.0 Hz, 1H). MS (ESI) m/z: 215 (M+H)⁺.

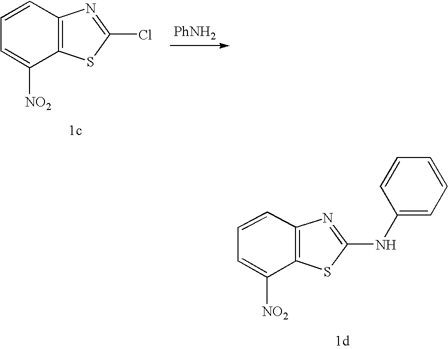

Using the procedure of Example 2 for preparing Compound 2a, Compound 1c (50 mg, 0.23 mmol), aniline (0.434 g, 4.66 mmol) and K₂CO₃ (64 mg, 0.46 mmol) were used to generate 15 mg (23%) of (7-nitro-benzothiazol-2-yl)-phenyl-amine Compound 1d. ¹H NMR (400 MHz, CDCl₃) δ 8.04 (d, J=7.8 Hz, 1H), 7.80 (d, J=7.9 Hz, 1H), 7.60-7.20 (m, 4H), 7.15 (m, 2H). MS (ESI) m/z: 272 (M+H)⁺.

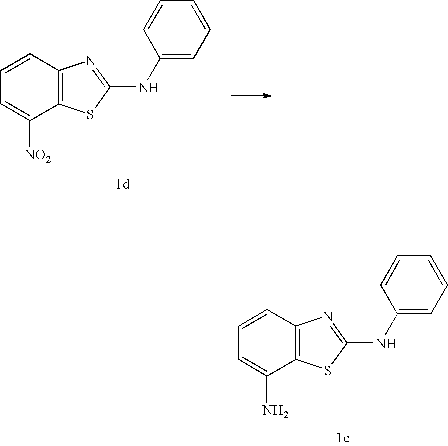

Using the procedure of Example 2 for preparing Compound 2b, Compound 1d (50 mg, 0.18 mmol), iron powder (31 mg, 0.54 mmol) and acetic acid (5 mL) were used to generate 36 mg (82%) of N²-phenyl-benzothiazole-2,7-diamine Compound 1e. ¹H NMR (300 MHz, CDCl₃) δ 7.45 (m, 4H), 7.15 (m, 2H), 7.05 (d, J=7.6 Hz, 4H), 6.52 (d, J=7.8 Hz, 2H). MS (ESI) m/z: 242 (M+H)⁺.

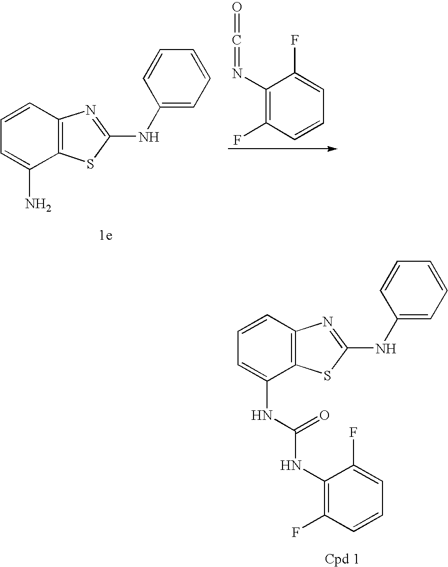

To a flask was added Compound 1e (6 mg, 1 equiv), 2,6-difluoro-phenyl-isocyanate (also referred to as 1,3-difluoro-2-isocyanato-benzene) (3.9 mg, 1 equiv) and CH₂Cl₂ and the mixture was stirred at rt for 2 hrs. The solvent was removed in vacuo and the residue was purified by flash chromatography to generate 4 mg (41%) of 1-(2,6-difluoro-phenyl)-3-(2-phenylamino-benzothiazol-7-yl)-urea Compound 1. ¹H NMR (300 MHz, CDCl₃) δ 7.55 (m, 5H), 7.15 (m, 3H), 6.95 (m, 3H). MS (ESI) m/z: 397 (M+H)⁺.

Using the procedure of Example 1, other compounds representative of the present invention were prepared:

| Cpd | Name and Data |
|---|---|
| 3 | 1-phenyl-3-(2-phenylamino-benzothiazol-7-yl)-urea |
| | ¹H NMR (300 MHz, CDCl₃) δ 11.95 (br s, |
| | 1H), 7.68 (d, J = 7.4 Hz, 2H), 7.56 (d, |
| | J = 7.6 Hz, 2H), 7.54 (d, J = 7.2 Hz, 2H), |
| | 7.35 (t, J = 7.4 Hz, 2H), 7.26 (t, J = 7.4 Hz, |
| | 2H), 7.04 (t, J = 7.4 Hz, 1H), 6.56 (d, |
| | J = 7.4 Hz, 1H). MS (ESI) m/z: |
| | 361 (M + H)⁺ |
| 4 | 1-(3,4-dichloro-phenyl)-3-(2-phenylamino-benzothiazol-7-yl)-urea |
| | ¹H NMR (300 MHz, CDCl₃) δ 12.30 (br s, |
| | 1H), 7.88 (s, 1H), 7.55 (m, 2H), |
| | 7.42 (m, 3H), 7.40 (d, J = 8.0 Hz, 1), 7.24 (t, |
| | J = 7.4 Hz, 2H), 6.60 (d, J = 7.6 Hz, 1H). |
| | MS (ESI) m/z: 429 (M + H)⁺ |

### EXAMPLE 2

### 1-phenyl-3-(2-phenylamino-benzothiazol-4-yl)-urea Compound 5

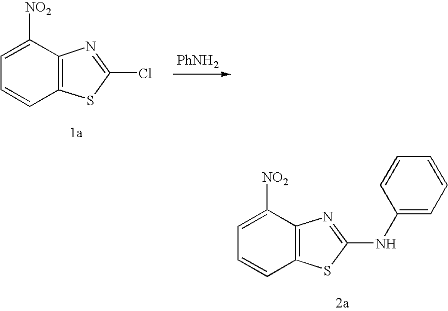

To a flask was added 2-chloro-4-nitro-benzothiazole Compound 1a (0.1 g, 0.47 mmol) (1 equiv), aniline (0.86 g, 9.23 mmol) (20 equiv), K₂CO₃ (0.13 g, 0.94 mmol) (2 equiv), THF and IPA. The mixture was refluxed for 2 days. The solvent was removed in vacuo and the residue was dissolved in CH₂Cl₂, then washed with H₂O. The organic layer was dried over MgSO₄, then concentrated and the residue was purified by flash chromatography (silica gel, CH₂Cl₂) to generate 68 mg (54%) of (4-nitro-benzothiazol-2-yl)-phenyl-amine Compound 2a. ¹H NMR (400 MHz, CDCl₃) δ 8.02 (d, J=7.6 Hz, 1H), 7.65 (d, J=7.6 Hz, 1H), 7.30 (m, 4H), 7.10 (m, 2H). MS (ESI) m/z: 272 (M+H)⁺.

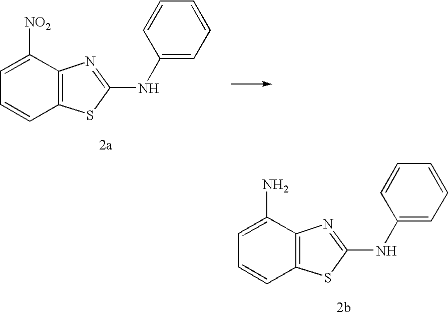

To a flask was added Compound 2a (100 mg, 0.37 mmol), iron powder (62 mg, 1.11 mmol) and acetic acid (6 mL). The mixture was heated to 75° C. for 2 hrs, then filtered. The filtrate was concentrated and the residue was purified by flash chromatography (silica gel, CH₂Cl₂:EtOAc/9.5:0.5) to afford 63 mg (71%) of N²-phenyl-benzothiazole-2,4-diamine Compound 2b. ¹H NMR (300 MHz, CDCl₃) δ 7.46 (d, J=7.6 Hz, 2H), 7.38 (t, J=7.8, 2H), 7.10 (t, J=7.6 Hz, 1H), 7.02 (m, 2H), 6.65 (d, J=7.4 Hz, 1H). MS (ESI) m/z: 242 (M+H)⁺.

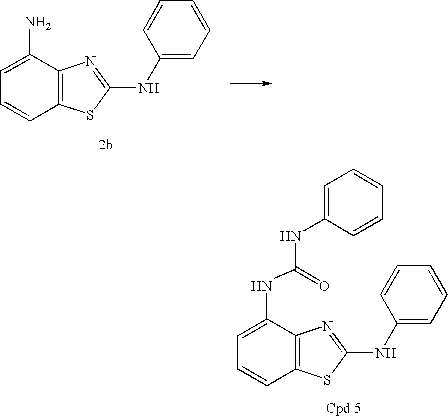

Using the procedure of Example 1 for preparing Compound 1, Compound 2b (6 mg) and phenyl-isocyanate (also referred to as isocyanato-benzene) (3 mg) were used to generate 8.2 mg (92%) of 1-phenyl-3-(2-phenylamino-benzothiazol-4-yl)-urea Compound 5. ¹H NMR (300 MHz, CDCl₃) δ 7.58 (m, 5H), 7.42 (d, J=7.2 Hz, 2H), 7.31 (t, J=7.0 Hz, 2H), 7.11 (d, J=7.4 Hz, 1H), 7.06 (d, J=7.2 Hz, 1H), 6.94 (d, J=7.2 Hz, 1H), 6.75 (d, J=7.4 Hz, 1H). MS (ESI) m/z: 361 (M+H)⁺.

Using the procedure of Example 2, other compounds representative of the present invention were prepared:

| Cpd | Name and Data |
|---|---|
| 6 | 1-(2,6-difluoro-phenyl)-3-(2-phenylamino-benzothiazol-4-yl)-urea |
| | ¹H NMR (300 MHz, CDCl₃) δ 7.55 (m, 5H), |
| | 7.25 (m, 1H), 7.04 (t, J = 7.2 Hz, 1H), |
| | 6.96 (m, 3H), 6.78 (d, J = 7.4 Hz, 1H). |
| | MS (ESI) m/z: 397 (M + H)⁺ |
| 7 | 1-(2-fluoro-phenyl)-3-(2-phenylamino-benzothiazol-4-yl)-urea |
| | ¹H NMR (300 MHz, CDCl₃) δ 12.55 (br s, |
| | 1H), 8.41 (t, J = 7.2 Hz, 1H), 7.58 (m, |
| | 3H), 7.41 (d, J = 7.2 Hz, 2H), 7.12 (m, 2H), |
| | 7.02 (m, 2H), 6.95 (d, J = 7.0 Hz, 1H), |
| | 6.75 (d, J = 7.6 Hz, 1H). MS (ESI) m/z: |
| | 379 (M + H)⁺ |
| 8 | 1-(2,6-dichloro-phenyl)-3-(2-phenylamino-benzothiazol-4-yl)-urea |
| | ¹H NMR (300 MHz, CDCl₃) δ 7.45 (m, 5H), |
| | 7.26 (d, J = 7.4 Hz, 2H), 7.05 (t, J = 7.4 Hz, |
| | 2H), 6.92 (t, J = 7.6 Hz, 1H), 6.84 (d, |
| | J = 7.6 Hz, 1H), 6.62 (d, J = 7.2 Hz, |
| | 1H). MS (ESI) m/z: 429 (M + H)⁺ |
| 60 | 1-tert-butyl-3-(2-phenylamino-benzothiazol-4-yl)-urea |
| | ¹H NMR (400 MHz, CDCl₃) 9.18 (s, |
| | 1H), 7.50 (m, 3H), 7.30 (d, 2H), 6.95 (m, 2H), |
| | 6.70 (d, 1H). MS (ESI) m/z: 341 (M + H)⁺ |
| 61 | 1-cyclohexyl-3-(2-phenylamino-benzothiazol-4-yl)-urea |
| | ¹H NMR (400 MHz, CDCl₃) 9.40 (s, |
| | 1H), 7.50 (m 3H), 7.35 (d, 2H), 6.98 (t, 2H), |
| | 6.65 (m, 1H), 2.00 (m, 2H), 1.70 (m, 2H), |
| | 1.60-1.10 (m, 7H). MS (ESI) m/z: |
| | 367 (M + H)⁺ |

### EXAMPLE 3

### 1-(2,6-difluoro-phenyl)-3-(2-phenylamino-benzothiazol-6-yl)-urea Compound 2

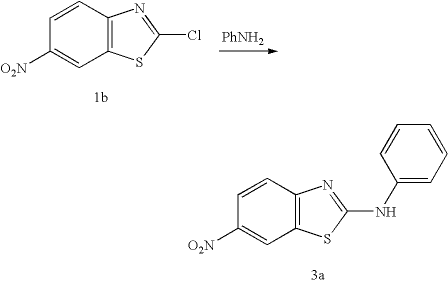

Using the procedure of Example 2 for preparing Compound 2a, 2-chloro-6-nitro-benzothiazole Compound 1b (0.1 g, 0.47 mmol), aniline (0.86 g, 9.23 mmol) and K₂CO₃ (0.13 g, 0.94 mmol) were used to generate 53 mg (42%) of (6-nitro-benzothiazol-2-yl)-phenyl-amine Compound 3a. ¹H NMR (400 MHz, CDCl₃) δ 8.38 (s, 1H), 7.96 (d, J=7.9 Hz, 1H), 7.51 (d, J=7.9 Hz, 1H), 7.46-7.30 (m, 4H), 7.12 (m, 1H). MS (ESI) m/z: 272 (M+H)⁺.

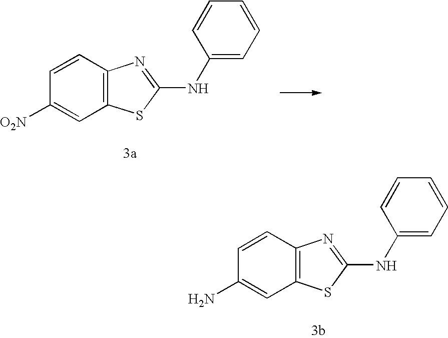

To a Parr flask was added Compound 3a (50 mg, 0.18 mmol), Raney nickel (11 mg, 0.18 mmol) and acetic acid (6 mL). The mixture was hydrogenated at 50 psi H₂ for 4 hrs, then filtered. The filtrate was concentrated and the residue was purified by flash chromatography (silica gel, CH₂Cl₂:EtOAc/7:3) to afford 32 mg (72%) of N²-phenyl-benzothiazole-2,6-diamine Compound 3b. ¹H NMR (300 MHz, CDCl₃) δ 7.35 (m, 5H), 7.02 (t, J=7.4 Hz, 1H), 6.85 (s, H), 6.60 (d, J=7.6 Hz, 1H). MS (ESI) m/z: 242 (M+H)⁺.

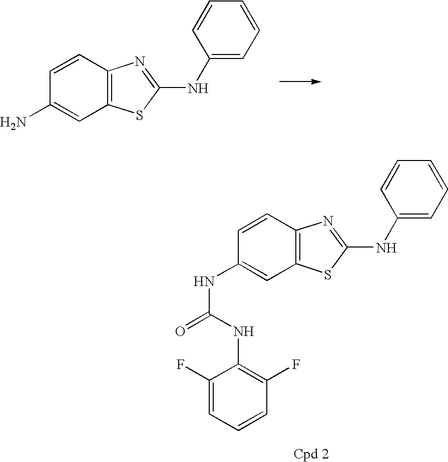

Using the procedure of Example 1 for preparing Compound 1, Compound 3b (10.0 mg) and 2,6-difluoro-phenyl-isocyanate (6.4 mg) were used to generate 9.2 mg (56%) of 1-(2,6-difluoro-phenyl)-3-(2-phenylamino-benzothiazol-6-yl)-urea Compound 2. ¹H NMR (300 MHz, CDCl₃) δ 11.05 (br s, 1H), 7.50 (m, 6H), 7.05 (m, 1H), 6.84 (t, J=7.6 Hz, 2H), 6.76 (s, 1H), 6.64 (d, J=7.6 Hz, 1H). MS (ESI) m/z: 397 (M+H)⁺.

### EXAMPLE 4

### N-(2-phenylamino-benzothiazol-4-yl)-benzamide Compound 9

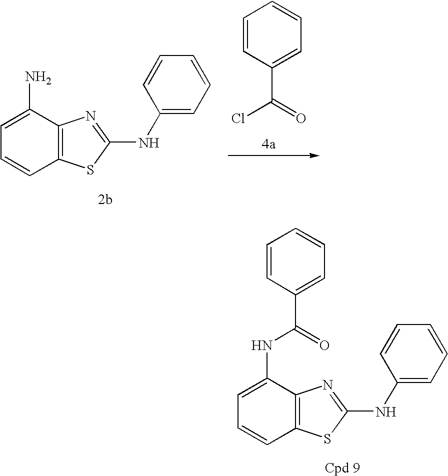

To a flask was added N²-phenyl-benzothiazole-2,4-diamine Compound 1g (5.0 mg, 0.021 mmol) (1 equiv), benzoyl chloride Compound 4a (2.9 mg, 0.021 mmol) (1.0 equiv), TEA (4.2 mg, 0.042 mmol) (2.0 equiv) and CH₂Cl₂. The mixture was stirred at rt for 2 hrs, then concentrated and the residue was purified by flash chromatography to generate 6.2 mg (86%) of N-(2-phenylamino-benzothiazol-4-yl)-benzamide Compound 9. ¹H NMR (300 MHz, CDCl₃) δ 9.25 (s, 1H), 8.52 (d, J=7.4 Hz, 1H), 8.02 (d, J=7.4 Hz, 2H), 7.50 (m, H), 7.32 (m, 3H), 7.12 (m, 2H). MS (ESI) m/z: 346 (M+H)⁺.

Using the procedure of Example 4, other compounds representative of the present invention were prepared:

| Cpd | Name and Data |
|---|---|
| 10 | 2,6-difluoro-N-(2-phenylamino-benzothiazol-4-yl)-benzamide |
| | ¹H NMR (300 MHz, CDCl₃) δ 8.94 (s, 1H), 8.46 (d, J = 7.4 Hz, 1H), 7.46 (d, J = 7.4 Hz, |
| | 2H), 7.40 (m, 1H), 7.38 (t, J = 7.6 Hz, 3H), 7.24 (t, J = 7.4 Hz, 1H), |
| | 7.16 (t, J = 7.2 Hz, 1H), 7.02 (t, J = 7.6 Hz, 2H). MS (ESI) m/z: 346 (M + H)⁺ |
| 13 | 2,5-dimethyl-2H-pyrazole-3-carboxylic acid (2-phenylamino-benzothiazol-4-yl)- |
| | amide |
| | ¹H NMR (300 MHz, CDCl₃) δ 7.52 (d, J = 7.2 Hz, 2H), 7.41 (t, J = 7.2 Hz, 2H), |
| | 7.10 (t, J = 7.3 Hz, 2H), 6.98 (m, 3H), 6.68 (d, J = 7.2 Hz, 1H), 4.25 (s, 6H). MS |
| | (ESI) m/z: 364 (M + H)⁺ |
| 19 | 3-nitro-N-(2-phenylamino-benzothiazol-4-yl)-benzamide |
| | ¹H NMR (400 MHz, CDCl₃) δ 11.92 (s, 1H), 9.28 (s, 1H), 8.82 (s, 1H), 8.46 (d, J = 6.8 Hz, |
| | 1H), 8.40 (d, J = 6.8 Hz, 1H), 8.35 (d, J = 6.9 Hz, 1H), 7.70 (t, J = 7.0 Hz, |
| | 1H), 7.55 (d, J = 7.0 Hz, 2H), 7.45 (d, J = 7.2 Hz, 1H), 7.40 (d, J = 7.2 Hz, 1H), |
| | 7.20 (m, 3H). MS (ESI) m/z: 391 (M + H)⁺ |
| 26 | N-(2-phenylamino-benzothiazol-4-yl)-propionamide |
| | ¹H NMR (400 MHz, CDCl₃) δ 8.35 (m, 2H), 7.45 (d, J = 7.2 Hz, 2H), 7.35 (t, J = 7.2 Hz, |
| | 2H), 7.28 (d, J = 7.1 Hz, 1H), 7.15 (m, 3H), 2.52 (q, J = 7.0 Hz, 2H), |
| | 1.30 (t, J = 7.0 Hz, 3H). MS (ESI) m/z: 298 (M + H)⁺ |
| 27 | pyridine-2-carboxylic acid (2-phenylamino-benzothiazol-4-yl)-amide |
| | ¹H NMR (400 MHz, CDCl₃) δ 11.15 (s, 1H), 8.75 (d, J = 4.0 Hz, 1H), 8.58 (d, J = 7.0 Hz, |
| | 1H), 8.32 (d, J = 7.0 Hz, 1H), 7.92 (t, J = 7.2 Hz, 1H), 7.60 (d, J = 7.2 Hz, |
| | 2H), 7.45 (t, J = 7.2 Hz, 1H), 7.40 (t, J = 7.2 Hz, 2H), 7.38 (d, J = 7.2 Hz, 1H), |
| | 7.20 (d, J = 7.0 Hz, 1H), 7.15 (t, J = 7.0 Hz, 1H). MS (ESI) m/z: 347 (M + H)⁺ |
| 28 | N-(2-phenylamino-benzothiazol-4-yl)-isonicotinamide |
| | ¹H NMR (400 MHz, CDCl₃) δ 9.25 (s, 1H), 8.82 (d, J = 6.2 Hz, 2H), 8.49 (d, J = 7.2 Hz, |
| | 1H), 7.82 (d, J = 6.2 Hz, 2H), 7.46 (d, J = 7.2 Hz, 2H), 7.40 (t, J = 7.2 Hz, |
| | 2H), 7.20 (m, 2H), 7.15 (d, J = 7.2 Hz, 1H). MS (ESI) m/z: 347 (M + H)⁺ |
| 29 | benzo[1,3]dioxole-5-carboxylic acid (2-phenylamino-benzothiazol-4-yl)-amide |
| | ¹H NMR (400 MHz, CDCl₃) δ 9.12 (s, 1H), 8.45 (d, J = 7.2 Hz, 1H), 7.50 (m, 3H), |
| | 7.35 (m, 3H), 7.30-6.90 (m, 6H), 6.70 (d, J = 7.0 Hz, 1H), 6.05 (s, 2H). MS |
| | (ESI) m/z: 390 (M + H)⁺ |
| 30 | N-(2-phenylamino-benzothiazol-4-yl)-benzenesulfonamide |
| | ¹H NMR (300 MHz, CDCl₃) δ 7.86 (d, J = 7.2 Hz, 2H), 7.60 (s, 1H), 7.52 (d, J = 7.1 Hz, |
| | 1H), 7.45-7.20 (m, 9H), 7.15 (m, 1H), 7.10 (t, J = 7.2 Hz, 1H). MS (ESI) |
| | m/z: 382 (M + H)⁺ |
| 31 | N-(2-phenylamino-benzothiazol-4-yl)-3,5-bis-trifluoromethyl-benzamide |
| | ¹H NMR (300 MHz, CDCl₃) δ 9.25 (s, 1H), 8.50 (d, J = 7.0 Hz, 1H), 8.45 (s, 2H), |
| | 8.10 (s, 1H), 7.52 (d, J = 7.2 Hz, 2H), 7.42 (m, 3H), 7.32 (m, 2H), 7.20 (t, J = 7.2 Hz, |
| | 1H). MS (ESI) m/z: 482 (M + H)⁺ |
| 32 | 2-bromo-N-(2-phenylamino-benzothiazol-4-yl)-benzamide |
| | ¹H NMR (400 MHz, CDCl₃) 9.00 (s, 1H), 8.55 (d, J = 7.0 Hz, 1H), 7.75 (d, J = 7.2 Hz, |
| | 1H), 7.65 (d, J = 7.2 Hz, 1H), 7.48 (d, J = 7.2 Hz, 2H), 7.40 (m, 4H), |
| | 7.20 (m, 1H), 7.10 (t, J = 7.2 Hz, 1H). MS (ESI) m/z: 426 (M + H)⁺ |
| 33 | 3-bromo-N-(2-phenylamino-benzothiazol-4-yl)-benzamide |
| | ¹H NMR (400 MHz, CDCl₃) 9.16 (s, 1H), 8.50 (d, J = 7.0 Hz, 1H), 8.13 (s, 1H), |
| | 7.95 (d, J = 7.2 Hz, 1H), 7.72 (d, J = 7.1 Hz, 1H), 7.55 (d, J = 7.1 Hz, 2H), |
| | 7.40 (m, 4H), 7.15 (m, 3H). MS (ESI) m/z: 426 (M + H)⁺ |
| 34 | biphenyl-4-carboxylic acid (2-phenylamino-benzothiazol-4-yl)-amide |
| | ¹H NMR (400 MHz, CDCl₃) 9.25 (s, 1H), 8.50 (d, J = 7.1 Hz, 1H), 8.02 (d, J = 7.2 Hz, |
| | 2H), 7.72 (d, J = 7.2 Hz, 2H), 7.62 (d, J = 7.1 Hz, 2H), 7.55-7.25 (m, 9H), |
| | 7.15 (m, 2H). MS (ESI) m/z: 422 (M + H)⁺ |
| 35 | thiophene-2-carboxylic acid (2-phenylamino-benzothiazol-4-yl)-amide |
| | ¹H NMR (400 MHz, CDCl₃) 9.08 (s, 1H), 8.45 (d, J = 7.2 Hz, 1H), 7.76 (d, J = 2.4 Hz, |
| | 1H), 7.60 (m, 4H), 7.45 (t, J = 7.2 Hz, 2H), 7.38 (d, J = 7.2 Hz, 1H), |
| | 7.20 (m, 3H). MS (ESI) m/z: 352 (M + H)⁺ |
| 36 | N-(2-phenylamino-benzothiazol-4-yl)-nicotinamide |
| | ¹H NMR (400 MHz, CDCl₃) 9.22 (m, 2H), 8.80 (s, 1H), 8.50 (d, J = 6.4 Hz, 1H), |
| | 8.30 (d, J = 6.4 Hz, 1H), 7.55-7.40 (m, 7H), 7.25 (m, 2H). MS (ESI) m/z: |
| | 347 (M + H)⁺ |
| 37 | furan-2-carboxylic acid (2-phenylamino-benzothiazol-4-yl)-amide |
| | ¹H NMR (400 MHz, CDCl₃) 9.40 (s, 1H), 8.45 (d, J = 7.0 Hz, 1H), 7.55 (m, 3H), |
| | 7.42 (d, J = 7.2 Hz, 2H), 7.40 (d, J = 7.2 Hz, 1H), 7.25 (m, 2H), 7.15 (m, 2H), |
| | 6.58 (m, 1H). MS (ESI) m/z: 336 (M + H)⁺ |
| 38 | 3-cyano-N-(2-phenylamino-benzothiazol-4-yl)-benzamide |
| | ¹H NMR (400 MHz, CDCl₃) 9.20 (s, 1H), 8.50 (s, 1H), 8.25 (m, 2H), 7.82 (m, 1H), |
| | 7.65 (m, 1H), 7.50 (m, 4H), 7.20 (m, 4H). MS (ESI) m/z: 371 (M + H)⁺ |
| 39 | 3,5-dimethyl-N-(2-phenylamino-benzothiazol-4-yl)-benzamide |
| | ¹H NMR (400 MHz, CDCl₃) 9.18 (s, 1H), 8.50 (d, J = 7.1 Hz, 1H), 7.55 (s, 2H), |
| | 7.50 (d, J = 7.2 Hz, 2H), 7.40 (t, J = 7.2 Hz, 2H), 7.35 (d, J = 7.1 Hz, 1H), |
| | 7.20 (m, 4H), 2.40 (s, 6H). MS (ESI) m/z: 374 (M + H)⁺ |
| 40 | naphthalene-2-carboxylic acid (2-phenylamino-benzothiazol-4-yl)-amide |
| | ¹H NMR (400 MHz, CDCl₃) 9.30 (s, 1H), 8.50 (s, J = 7.0 Hz, 1H), 8.40 (s, 1H), |
| | 7.95 (d, J = 7.2 Hz, 1H), 7.90 (t, J = 7.2 Hz, 2H), 7.82 (d, J = 7.2 Hz, 1H), |
| | 7.50 (m, 4H), 7.30 (m, 3H), 7.10 (m, 3H). MS (ESI) m/z: 396 (M + H)⁺ |
| 41 | cyclohexanecarboxylic acid (2-phenylamino-benzothiazol-4-yl)-amide |
| | ¹H NMR (300 MHz, CDCl₃) 8.50 (s, 1H), 8.39 (d, J = 7.2 Hz, 1H), 7.50 (d, J = 7.2 Hz, |
| | 2H), 7.40 (t, J = 7.2 Hz, 2H), 7.28 (d, J = 7.2 Hz, 1H), 7.15 (m, 3H), |
| | 2.35 (m, 1H), 2.05 (m, 2H), 1.85 (m, 2H), 1.80-1.50 (m, 3H), 1.45-1.15 (m, 3H). MS |
| | (ESI) m/z: 352 (M + H)⁺ |
| 42 | 5-ethyl-thiophene-2-carboxylic acid (2-phenylamino-benzothiazol-4-yl)-amide |
| | ¹H NMR (400 MHz, CDCl₃) 9.00 (s, 1H), 8.42 (d, J = 7.2 Hz, 1H), 7.60 (m, 3H), |
| | 7.40 (d, J = 7.2 Hz, 2H), 7.35 (d, J = 7.2 Hz, 1H), 7.28 (d, J = 7.2 Hz, 1H), |
| | 7.20 (m, 2H), 6.82 (d, J = 6.2 Hz, 1H), 2.95 (q, J = 6.8 Hz, 2H), 1.40 (t, J = 6.8 Hz, 3H). |
| | MS (ESI) m/z: 380 (M + H)⁺ |
| 43 | 3,5-dinitro-N-(2-phenylamino-benzothiazol-4-yl)-benzamide |
| | ¹H NMR (400 MHz, CDCl₃) 9.23 (s, 3H), 8.55 (s, 1H), 8.50 (d, J = 7.2 Hz, 1H), |
| | 7.45 (m, 4H), 7.20 (m, 4H). MS (ESI) m/z: 436 (M + H)⁺ |
| 44 | 2,4,6-trichloro-N-(2-phenylamino-benzothiazol-4-yl)-benzamide |
| | ¹H NMR (300 MHz, CDCl₃) 8.50 (s, 1H), 8.38 (d, J = 7.0 Hz, 1H), 7.30 (m, 9H), |
| | 7.10 (m, 1H). MS (ESI) m/z: 450 (M + H)⁺ |
| 45 | 2,4,6-trifluoro-N-(2-phenylamino-benzothiazol-4-yl)-benzamide |
| | ¹H NMR (300 MHz, CDCl₃) 8.96 (s, 1H), 8.45 (d, J = 7.2 Hz, 1H), 7.50 (d, J = 7.2 Hz, |
| | 2H), 7.40 (t, J = 7.2 Hz, 2H), 7.35-7.10 (m, 5H), 7.00 (m, 1H). MS (ESI) |
| | m/z: 400 (M + H)⁺ |
| 46 | 2,6-dimethoxy-N-(2-phenylamino-benzothiazol-4-yl)-benzamide |
| | ¹H NMR (300 MHz, CDCl₃) 8.70 (s, 1H), 8.56 (d, J = 7.2 Hz, 1H), 7.36 (d, J = 7.2 Hz, |
| | 2H), 7.26 (t, J = 7.2 Hz, 4H), 7.20 (m, 2H), 7.05 (t, J = 7.1 Hz, 1H), |
| | 6.55 (d, J = 7.1 Hz, 2H), 3.85 (s, 6H). MS (ESI) m/z: 406 (M + H)⁺ |
| 47 | 2,6-dichloro-N-(2-phenylamino-benzothiazol-4-yl)-benzamide |
| | ¹H NMR (400 MHz, CDCl₃) 8.50 (s, 1H), 8.45 (d, J = 7.2 Hz, 1H), 7.30 (m, 10H), |
| | 7.04 (t, J = 7.0 Hz, 1H). MS (ESI) m/z: 415 (M + H)⁺ |
| 48 | 2,6-difluoro-3-methyl-N-(2-phenylamino-benzothiazol-4-yl)-benzamide |
| | ¹H NMR (400 MHz, CDCl₃) 8.95 (s, 1H), 8.52 (d, J = 7.2 Hz, 1H), 7.50 (d, J = 7.2 Hz, |
| | 2H), 7.40 (t, J = 7.2 Hz, 2H), 7.30-7.20 (m, 4H), 7.15 (t, J = 7.1 Hz, 1H), |
| | 6.90 (t, J = 7.1 Hz, 1H), 2.30 (s, 3H). MS (ESI) m/z: 396 (M + H)⁺ |
| 49 | 2,6-difluoro-3-methyl-N-{2-[4-(4-methyl-piperazin-1-yl)-phenylamino]- |
| | benzothiazol-4-yl}-benzamide |
| | ¹H NMR (300 MHz, CDCl₃) 8.90 (s, 1H), 8.53 (d, J = 7.2 Hz, 1H), 8.95 (s, H), |
| | 8.52 (d, J = 7.2 Hz, 1H), 7.35 (m, 4H), 7.25 (m, 1H), 7.15 (t, J = 7.1 Hz, 1H), |
| | 6.90 (m, 3H), 3.20 (t, J = 6.2 Hz, 4H), 2.65 (t, J = 6.2 Hz, 4H), 2.40 (s, 3H). MS |
| | (ESI) m/z: 494 (M + H)⁺ |
| 50 | benzo[b]thiophene-2-carboxylic acid (2-phenylamino-benzothiazol-4-yl)-amide |
| | ¹H NMR (400 MHz, CDCl₃) 9.15 (s, 1H), 8.38 (d, J = 7.1 Hz, 1H), 7.90 (s, 1H), |
| | 7.82 (m, 2H), 7.50 (d, J = 7.2 Hz, 2H), 7.40 (m, 4H), 7.30 (d, J = 7.1 Hz, 1H), |
| | 7.15 (m, 4H). MS (ESI) m/z: 402 (M + H)⁺ |
| 51 | 2-phenyl-N-(2-phenylamino-benzothiazol-4-yl)-acetamide |
| | ¹H NMR (300 MHz, CDCl₃) 8.35 (s, 1H), 7.40-7.15 (m, 10H), 7.05 (m, 3H), |
| | 3.78 (s, 2H). MS (ESI) m/z: 360 (M + H)⁺ |
| 52 | cyclopentanecarboxylic acid (2-phenylamino-benzothiazol-4-yl)-amide |
| | ¹H NMR (400 MHz, CDCl₃) 8.45 (s, 1H), 8.35 (d, J = 6.8 Hz, 1H), 7.48 (d, J = 7.2 Hz, |
| | 2H), 7.40 (t, J = 7.2 Hz, 2H), 7.30 (d, J = 7.2 Hz, 1H), 7.15 (m, 2H), |
| | 2.85 (m, 1H), 2.00 (m, 2H), 1.85 (m, 1H), 1.70 (m, 1H). MS (ESI) m/z: 338 (M + H)⁺ |
| 53 | (2-phenylamino-benzothiazol-4-yl)-carbamic acid phenyl ester |
| | ¹H NMR (400 MHz, CDCl₃) 8.52 (s, 1H), 8.25 (d, J = 7.2 Hz, 1H), 7.45 (m, 8H), |
| | 7.35 (d, J = 7.2 Hz, 1H), 7.25 (m, 2H), 7.15 (d, J = 7.2 Hz). MS (ESI) m/z: |
| | 362 (M + H)⁺ |
| 54 | 3-phenyl-N-(2-phenylamino-benzothiazol-4-yl)-propionamide |
| | ¹H NMR (300 MHz, CDCl₃) 8.42 (d, J = 7.2 Hz, 1H), 8.36 (s, 1H), 7.50 (d, J = 7.1 Hz, |
| | 2H), 7.40 (t, J = 7.1 Hz, 2H), 7.30 (m, 6H), 7.15 (m, 3H). MS (ESI) m/z: |
| | 372 (M + H)⁺ |
| 55 | cycloheptanecarboxylic acid (2-phenylamino-benzothiazol-4-yl)-amide |
| | ¹H NMR (400 MHz, CDCl₃) 8.45 (s, 1H), 8.40 8.42 (d, J = 7.2 Hz, 1H), 7.50 (d, |
| | J = 7.2 Hz, 2H), 7.40 (t, J = 7.1 Hz, 2H), 7.30 (d, J = 7.2 Hz, 1H), 7.15 (m, 2H), |
| | 2.55 (m, 1H), 2.05 (m, 2H), 1.85 (m 4H), 1.65 (m, 6H). MS (ESI) m/z: 364 (M − H)⁺ |
| 56 | 4-methyl-[1,2,3]thiadiazole-5-carboxylic acid (2-phenylamino-benzothiazol-4-yl)- |
| | amide |
| | ¹H NMR (400 MHz, CD₃OD) 8.05 (s, 1H), 7.77 (d, J = 7.0 Hz, 2H), 7.55 (d, J = 7.0 Hz, |
| | 1H), 7.40 (t, J = 7.1 Hz, 2H), 7.20 (d, J = 7.0 Hz, 1H), 3.10 (s, 3H). MS |
| | (ESI) m/z: 366 (M − H)⁺ |
| 57 | 2,2-dimethyl-N-(2-phenylamino-benzothiazol-4-yl)-propionamide |
| | ¹H NMR (300 MHz, CDCl₃) 8.85 (s, 1H), 8.42 (d, J = 7.1 Hz, 1H), 7.60 (d, J = 7.2 Hz, |
| | 2H), 7.45 (t, J = 7.1 Hz, 2H), 7.32 (d, J = 7.1 Hz, 1H), 7.15 (m, 1H), |
| | 1.45 (s, 3H). MS (ESI) m/z: 324 (M − H)⁺ |
| 58 | 2-cyclohexyl-N-(2-phenylamino-benzothiazol-4-yl)-acetamide |
| | ¹H NMR (400 MHz, CDCl₃) 8.30 (m, 1H), 8.05 (m, 1H), 7.60 (d, 2H), 7.50 (m, 1H), |
| | 7.35 (m, 3H), 2.20 (m, 2H), 1.75 (m, 5H), 1.20 (m, 4H), 0.90 (m 2H). MS |
| | (ESI) m/z: 366 (M + H)⁺ |
| 59 | 4,6-dichloro-1H-indole-2-carboxylic acid (2-phenylamino-benzothiazol-4-yl)- |
| | amide |
| | ¹H NMR (400 MHz, CDCl₃) 12.35 (s, 1H), 10.65 (s, 1H), 10.05 9s, 1H), 7.80 (m, |
| | 3H), 7.60 (m, 1H), 7.40 (m, 1H), 7.25 (m, 2H), 7.15 (m, 1H), 7.05 (m, 1H). MS |
| | (ESI) m/z: 453 (M + H)⁺ |

### EXAMPLE 5

### 2,6-difluoro-N-[2-(4-methoxy-phenylamino)-benzothiazol-4-yl]-3-methyl-benzamide Compound 11

Using the procedure of Example 2 for preparing Compound 2a, 2-chloro-4-nitro-benzothiazole Compound 1a (0.2 g, 0.93 mmol), 4-methoxyaniline (0.2 g, 1.62 mmol), K₂CO₃ (0.26 g, 1.88 mmol), THF (10 mL) and IPA (10 mL) were used to generate 0.15 g (52%) of (4-methoxy-phenyl)-(4-nitro-benzothiazol-2-yl)-amine Compound 5a. ¹H NMR (400 MHz, CDCl₃) δ 7.98 (d, J=7.2 Hz, 1H), 7.68 (d, J=7.2 Hz, H), 7.22 (d, J=7.3 Hz, 2H), 7.02 (t, J=7.2 Hz, 1H), 6.85 (d, J=7.2 Hz, 2H), 3.75 (s, 3H). MS (ESI) m/z: 302 (M+H)⁺.

Using the procedure of Example 2 for preparing Compound 2a, Compound 5a (0.15 g, 0.50 mmol), iron powder (0.1 g, 1.79 mmol) and acetic acid (10 mL) were used to generate 81 mg (60%) of N²-(4-methoxy-phenyl)-benzothiazole-2,4-diamine Compound 5b. ¹H NMR (400 MHz, CDCl₃) δ 7.40 (d, J=7.2 Hz, 2H), 6.95 (m, 2H), 6.90 (d, J=7.4 Hz, 2H), 6.65 (d, J=7.2 Hz, 1H), 3.80 (s, 3H). MS (ESI) m/z: 272 (M+H)⁺.

Using the procedure of Example 4 for preparing Compound 9, Compound 5b (10 mg, 0.037 mmol), 2,6-difluoro-3-methylbenzoyl chloride (7.0 mg, 0.037 mmol) and pyridine were used to generate 13.5 mg (86%) of 2,6-difluoro-N-[2-(4-methoxy-phenylamino)-benzothiazol-4-yl]-3-methyl-benzamide Compound 11. ¹H NMR (400 MHz, CDCl₃) δ 8.95 (s, 1H), 8.47 (d, J=7.4 Hz, 1H), 7.41 (d, J=7.4 Hz, 2H), 7.40 (m, 1H), 7.25 (t, J=7.6 Hz, 3H), 7.16 (t, J=7.4 Hz, 1H), 7.11 (m, 1H), 6.88 (d, J=7.4 Hz, 2H), 6.85 (m, 1H), 3.80 (s, 3H), 2.30 (s, 3H). MS (ESI) m/z: 426 (M+H)⁺.

### EXAMPLE 6

### 2,6-difluoro-3-methyl-N-[2-(4-sulfamoyl-phenylamino)-benzothiazol-4-yl]-benzamide Compound 12

Using the procedure of Example 2 for preparing Compound 2a, 2-chloro-4-nitro-benzothiazole Compound 1a (0.1 g, 0.47 mmol), sulfanilamide (0.1 g, 0.59 mmol), K₂CO₃ (0.1 g), i-PrOH (10 ml) and THF (10 mL) were used to generate 82 mg (50%) of 4-(4-nitro-benzothiazol-2-ylamino)-benzenesulfonamide Compound 6a. ¹H NMR (300 MHz, MeOD) δ 8.12 (d, J=7.2 Hz, 1H), 7.88 (d, J=7.2 Hz, 1H), 7.60 (d, J=7.3 Hz, 2H), 7.25 (t, J=7.2 Hz, 1H), 6.66 (d, J=7.2 Hz, 2H). MS (ESI) m/z: 349 (M−H)⁺.

Using the procedure of Example 2 for preparing Compound 2b, Compound 6a (0.1 g, 0.29 mmol) was dissolved in MeOH, followed by addition of 10% Pd/C (15 mg). The mixture was hydrogenated under 30 psi H₂ for 2 hrs, then filtered. The filtrate was concentrated and the residue was purified by gravity chromatography (silica gel, CH₂Cl₂:MeOH/9.5:0.5) to afford 59 mg (64%) of 4-(4-amino-benzothiazol-2-ylamino)-benzenesulfonamide Compound 6b. ¹H NMR (300 MHz, MeOD) δ 7.60 (d, J=7.2 Hz, 2H), 6.96 (t, J=7.2 Hz, 1H), 6.88 (d, J=7.3 Hz, 1H), 6.68 (m, 3H). MS (ESI) m/z: 321 (M+H)⁺.

Using the procedure of Example 4 for preparing Compound 9, Compound 6b (10 mg, 0.031 mmol), 2,6-difluoro-3-methylbenzoyl chloride (4.1 mg, 0.022 mmol) and pyridine (20 μL) were used to generate 7.6 mg (51%) of 2,6-difluoro-3-methyl-N-[2-(4-sulfamoyl-phenylamino)-benzothiazol-4-yl]-benzamide Compound 12. ¹H NMR (300 MHz, MeOD) δ 7.80 (m, 1H), 7.62 (d, J=7.2 Hz, 2H), 7.50 (d, J=7.4 Hz, 2H), 7.40 (m, 1H), 7.26 (m, 1H), 7.02 (t, J=7.2 Hz, 1H), 6.62 (d, J=7.2 Hz, 1H), 2.32 (s, 3H). MS (ESI) m/z: 475 (M+H)⁺.

### EXAMPLE 7

### 2,6-difluoro-3-methyl-N-(2-p-tolylamino-benzothiazol-4-yl)-benzamide Compound 14

Using the procedure of Example 2 for preparing Compound 2a, 2-chloro-4-nitro-benzothiazole Compound 1a (0.20 g, 0.93 mmol), p-toluidine (0.20 g, 1.87 mmol) and K₂CO₃ (0.26 g, 1.87 mmol) were used to generate 0.16 g (61%) of (4-nitro-benzothiazol-2-yl)-p-tolyl-amine Compound 7a. ¹H NMR (300 MHz, CDCl₃) δ 8.10 (d, J=7.2 Hz, 1H), 7.80 (d, J=7.2 Hz, 1H), 7.30 (m, 5H), 2.40 (s, 3H). MS (ESI) m/z: 284 (M−H)⁺.

To a Parr flask was added Compound 7a (0.15 g), 10% Pd/C (50 mg) and MeOH (10 mL). The mixture was hydrogenated under 30 psi H₂ for 2 hrs, then filtered. The filtrate was concentrated to afford 86 mg (64%) of N²-p-tolyl-benzothiazole-2,4-diamine Compound 7b. ¹H NMR (300 MHz, CDCl₃) δ 7.40 (d, J=7.2 Hz, 2H), 7.21 (d, J=7.2 Hz, 2H), 7.00 (m, 2H), 6.69 (d, J=7.0 Hz, 1H), 2.40 (s, 3H). MS (ESI) m/z: 256 (M+H)⁺.

Using the procedure of Example 4 for preparing Compound 9, Compound 7b (2 mg, 0.0078 mmol), 2,6-difluoro-3-methylbenzoyl chloride (1 mg, 0.0052 mmol) and pyridine (10 μL) were used to generate 1.3 mg (40%) of 2,6-difluoro-3-methyl-N-(2-p-tolylamino-benzothiazol-4-yl)-benzamide Compound 14. ¹H NMR (300 MHz, CDCl₃) δ 8.96 (br s, 1H), 8.46 (d, J=8.0 Hz, 1H), 7.40-7.10 (m, 8H), 6.90 (t, J=7.4 Hz, 1H), 2.34 (s, 3H), 2.32 (s, 3H). MS (ESI) m/z: 410 (M+H)⁺.

### EXAMPLE 8

### pyrimidine-4-carboxylic acid (2-phenylamino-benzothiazol-4-yl)-amide Compound 15

To a flask was added N²-phenyl-benzothiazole-2,4-diamine Compound 2b (3 mg, 0.012 mmol), pyrimidine-4-carboxylic acid (1.5 mg, 0.012 mmol), HOBt (1.7 mg, 0.012 mmol), DIC (1.6 mg, 0.012 mmol) and DMF (2 mL). The mixture was stirred at rt overnight, then poured into H₂O and extracted with EtOAc. The organic layer was dried over MgSO₄, then concentrated and the residue was purified by flash chromatography (silica gel, CH₂Cl₂:EtOAc/5:5) to afford 3.5 mg (82%) of pyrimidine-4-carboxylic acid (2-phenylamino-benzothiazol-4-yl)-amide Compound 15. ¹H NMR (400 MHz, DMSO) δ 10.95 (s, 1H), 10.65 (s, 1H), 9.35 (s, 1H), 9.02 (m, 1H), 8.90 (s, 1H), 8.35 (d, J=7.1 Hz, 1H), 7.88 (d, J=7.2 Hz, 2H), 7.58 (d, J=7.2 Hz, 1H), 7.45 (t, J=7.2 Hz, 2H), 7.22 (t, J=7.2 Hz, 1H), 7.12 (t, J=7.2 Hz, 1H). MS (ESI) m/z: 348 (M+H)⁺.

Using the procedure of Example 8, other compounds representative of the present invention were prepared:

| Cpd | Name and Data |
|---|---|
| 21 | 6-morpholin-4-yl-N-(2-phenylamino- |
| | benzothiazol-4-yl)-nicotinamide |
| | ¹H NMR (400 MHz, CD₃COCD₃) δ 9.71 (s, |
| | 1H), 9.25 (s, 1H), 8.84 (s, 1H), |
| | 8.38 (d, J = 6.8 Hz, 1H), 8.12 (d, J = 7.0 Hz, 1H), |
| | 7.86 (d, J = 7.2 Hz, 2H), 7.48 (d, J = 6.8 Hz, |
| | 1H), 7.45 (d, J = 7.0 Hz, 2H), 7.16 (t, |
| | J = 7.0 Hz, 1H), 7.08 (t, J = 7.1 Hz, |
| | 1H), 6.95 (d, J = 7.0 Hz, 1H), 3.79 (m, |
| | 4H), 3.70 (m, 4H). MS (ESI) m/z: |
| | 432 (M + H)⁺ |

### EXAMPLE 9

### N-(2-phenylamino-benzothiazol-4-yl)-acetamide Compound 16

Adapting the procedure of Example 8, N²-phenyl-benzothiazole-2,4-diamine Compound 2b (10 mg, 0.041 mmol), 3-acetoxybenzoic acid (7.5 mg, 0.041 mmol), HOBt (5.6 mg, 0.041 mmol) and DIC (5.2 mg, 0.041 mmol) were used to generate 7.5 mg (64%) of N-(2-phenylamino-benzothiazol-4-yl)-acetamide Compound 16. ¹H NMR (400 MHz, CDCl₃) δ 8.35 (m, 2H), 7.47 (t, J=7.2 Hz, 2H), 7.41 (t, J=7.1 Hz, 1H), 7.32 (d, J=7.2 Hz, 1H), 7.16 (t, J=7.2 Hz, 2H), 2.26 (s, 3H). MS (ESI) m/z: 284 (M+H)⁺.

Using the procedure of Example 9, other compounds representative of the present invention were prepared:

| Cpd | Name and Data |
|---|---|
| 17 | N-(2-phenylamino-benzothiazol-4-yl)-4-sulfamoyl-benzamide |
| | ¹H NMR (400 MHz, DMSO) δ 10.65 (s, 1H), 10.12 (s, 1H), 8.21 (d, J = 7.3 Hz, 2H), |
| | 8.04 (d, J = 7.2 Hz, 2H), 7.88 (d, J = 7.2 Hz, 3H), 7.68 (d, J = 7.3 Hz, 1H), |
| | 7.56 (s, 1H), 7.34 (t, J = 7.2 Hz, 2H), 7.20 (t, J = 7.2 Hz, 1H), 7.05 (t, J = 7.05 Hz, |
| | 1H). MS (ESI) m/z: 425 (M + H)⁺ |
| 18 | isoxazole-5-carboxylic acid (2-phenylamino-benzothiazol-4-yl)-amide |
| | ¹H NMR (400 MHz, CDCl₃) δ 9.58 (s, 1H), 8.42 (m, 2H), 7.52 (d, J = 7.0 Hz, 2H), |
| | 7.42 (m, 2H), 7.20 (m, 4H), 7.05 (s, 1H). MS (ESI) m/z: 337 (M + H)⁺ |
| 22 | N-(2-phenylamino-benzothiazol-4-yl)-2-tetrazol-1-yl-acetamide |
| | ¹H NMR (400 MHz, CD₃COCD₃) δ 9.59 (s, 1H), 9.45 (s, 1H), 9.25 (s, 1H), |
| | 8.18 (d, J = 7.2 Hz, 1H), 7.80 (d, J = 7.2 Hz, 2H), 7.52 (d, J = 7.0 Hz, 1H), 7.38 (t, J = 7.2, |
| | 2H), 7.15 (t, J = 7.2 Hz, 1H), 7.05 (t, J = 7.2 Hz, 1H), 6.78 (s, 2H). MS |
| | (ESI) m/z: 352 (M + H)⁺ |
| 23 | 2-(3,5-difluoro-phenyl)-N-(2-phenylamino-benzothiazol-4-yl)-acetamide |
| | ¹H NMR (400 MHz, CDCl₃) δ 8.38 (s, 1H), 8.32 (d, J = 7.1 Hz, 1H), 7.41 (m, 4H), |
| | 7.36 (d, J = 7.2 Hz, 2H), 7.15 (m, 3H), 6.95 (d, J = 7.2 Hz, 2H), 6.75 (t, J = 7.2 Hz, |
| | 1H), 3.78 (s, 2H). MS (ESI) m/z: 396 (M + H)⁺ |
| 24 | 5-bromo-N-(2-phenylamino-benzothiazol-4-yl)-nicotinamide |
| | ¹H NMR (400 MHz, CD₃COCD₃) δ 9.66 (s, 1H), 9.94 (s, 1H), 9.18 (s, 1H), |
| | 8.85 (s, 1H), 8.58 (s, 1H), 8.32 (d, J = 7.2 Hz, 1H), 7.85 (d, J = 7.2 Hz, 2H), 7.52 (d, J = 7.2 Hz, |
| | 1H), 7.40 (t, J = 7.2 Hz, 2H), 7.15 (t, J = 7.1 Hz, 1H), 7.05 (t, J = 7.1 Hz, |
| | 1H). MS (ESI) m/z: 426 (M + H)⁺ |
| 25 | 1-acetyl-piperidine-4-carboxylic acid (2-phenylamino-benzothiazol-4-yl)-amide |
| | ¹H NMR (400 MHz, CDCl₃) δ 8.50 (s, 1H), 8.36 (d, J = 7.2 Hz, 1H), 8.04 (s, 1H), |
| | 7.81 (s, 1H), 7.52 (d, J = 7.2 Hz, 2H), 7.42 (t, J = 7.2 Hz, 2H), 7.35 (d, J = 7.2 Hz, |
| | 1H), 7.15 (m, 2H), 4.70 (m, 1H), 3.96 (m, 1H), 3.18 (m, 1H), 2.80 (m, 1H), |
| | 2.62 (m, 1H), 2.05 (m, 1H), 1.85 (m, 1H). MS (ESI) m/z: 395 (M + H)⁺ |

### EXAMPLE 10

### 3-amino-N-(2-phenylamino-benzothiazol-4-yl)-benzamide Compound 20

3-nitro-N-(2-phenylamino-benzothiazol-4-yl)-benzamide Compound 19 (15 mg 0.038 mmol) was dissolved in acetic acid and iron powder (15 mg, 0.27 mmol) was added. The mixture was purged with N₂, then heated to 70° C. for 1 hr. The solvent was removed in vacuo and the residue was purified by flash chromatography (silica gel, CH₂Cl₂:EtOAc/8:2) to afford 8.3 mg (60%) of 3-amino-N-(2-phenylamino-benzothiazol-4-yl)-benzamide Compound 20. ¹H NMR (400 MHz, CDCl₃) δ 8.95 (s, 1H), 8.40 (d, J=7.0 Hz, 1H), 7.42 (d, J=7.2 Hz, 2H), 7.30 (m, 3H), 7.22 (d, J=7.1 Hz, 1H), 7.15 (d, J=7.1 Hz, 1H), 7.10 (t, J=7.0 Hz, 1H), 6.78 (d, J=7.0 Hz, 1H). MS (ESI) m/z: 361 (M+H)⁺.

### EXAMPLE 11

### 2,6-difluoro-3,N-dimethyl-N-(2-phenylamino-benzothiazol-4-yl)-benzamide Compound 62

To a flask was added 2,6-difluoro-3-methyl-N-(2-p-tolylamino-benzothiazol-4-yl)-benzamide Compound 14 (11 mg, 0.028 mmol) and DMF (1 mL), followed by addition of NaH (1.1 mg, 60% dispersion in mineral oil). After 10 minutes, methyl iodide was added and the mixture was stirred overnight. The reaction mixture was poured into H₂O and extracted with EtOAc. The organic layer was dried over MgSO4, then concentrated and the residue was purified by flash chromatography (silica gel, CH₂Cl₂) to afford 10 mg (90%) of 2,6-difluoro-3,N-dimethyl-N-(2-phenylamino-benzothiazol-4-yl)-benzamide Compound 62. ¹H NMR (400 MHz, CDCl₃) δ 8.96 (s, 1H), 8.42 (d, J=7.2 Hz, 1H), 7.42 (m, 3H), 7.32 (m, 1H), 7.25 (m, 3H), 7.10 (t, J=7.2 Hz, 1H), 6.90 (t, J=7.2 Hz, 1H), 3.60 9s, 3H), 2.32 (s, 3H). MS (ESI) m/z: 410 (M+H)⁺.

### BIOLOGICAL EXAMPLES

The ability of the compounds for treating a chronic or acute kinase mediated disease, disorder or condition was determined using the following procedures.

### EXAMPLE 1

### CDK1 Screening Assay

A kinase reaction mixture was prepared containing 50 mM Tris.HCl pH=8, 10 mM MgCl₂, 0.1 mM Na₃PO₄, 1 mM DTT, 10 μM ATP, 0.025 μM biotinylated histone-H1 peptide substrate and 0.2 μCuries per well ³³P-γ-ATP (2000-3000 Ci/mmol). 70 μL of the kinase reaction mixture was dispensed into the well of a streptavidin coated FlashPlate™ (Cat. # SMP103, NEN, Boston, Mass.). Then 1 μL of test compound stock in 100% DMSO was added to the wells resulting in a final concentration of 1% DMSO in the reaction well with a 100 μL final reaction well volume.

The CDK1:Cyclin-B protein was diluted in 50 mM Tris.HCl pH=8.0, 0.1% BSA at a concentration of 1 ng per μL and 30 μL (30 ng enzyme per test well) was added to each well to initiate the reaction. The reaction was incubated for one hour at 30° C. At the end of the one hour incubation, the reaction was terminated by aspirating the mixture from the plate and washing the wells twice with PBS containing 100 mM EDTA. The histone-H1 biotinylated peptide substrate became immobilized on the Flashplate™ and the incorporation of ³³P-γ-ATP was measured by reading the plate on a scintillation counter. Inhibition of the enzymatic activity of CDK1 was measured by observing a reduced amount of ³³P-γ-ATP incorporated into the immobilized peptide.

The CDK1 kinase used was isolated from insect cells expressing both the human CDK1 catalytic subunit and its positive regulatory subunit cyclin B (New England Biolabs, Beverly, Mass., Cat. # 6020).

### EXAMPLE 2

### VEGF-R2 Screening Assay

A kinase reaction mixture was prepared containing 50 mM Tris.HCl pH=8, 10 mM MgCl₂, 0.1 mM Na₃PO₄, 1 mM DTT, 10 μM ATP, 0.025 μM biotinylated peptide substrate and 0.8 μCuries per well ³³P-γ-ATP (2000-3000 Ci/mmol). 70 μL of the kinase reaction mixture was dispensed into the well of a streptavidin coated FlashPlate™ (Cat. # SMP103, NEN, Boston, Mass.). Then 1 μL of test compound stock in 100% DMSO was added to the wells resulting in a final concentration of 1% DMSO in the reaction well with a 100 μL final reaction well volume.

The soluble rat tyrosine kinase containing an N-terminal 6×HIS tag was diluted in 50 mM Tris.HCl pH=8.0, 0.1% BSA at a concentration of 5 ng per μL and 30 μL (150 ng enzyme per test well) was added to each well to initiate the reaction. The reaction was incubated for one hour at 30° C. At the end of the one hour incubation, the reaction was terminated by aspirating the reaction mixture from the plate and washing the wells twice with PBS containing 100 mM EDTA. The PLC1 biotinylated peptide substrate became immobilized on the Flashplate™ and the incorporation of ³³P-γ-ATP was measured by reading the plate on a scintillation counter. Inhibition of the enzymatic activity of the VEGF-R was measured by observing a reduced amount of ³³P-γ-ATP incorporated into the immobilized peptide.

The VEGF-R kinase assay was carried out using the CDK kinase assay procedure except that the enzyme was replaced with the VEGF-R2 fusion protein containing a polyhistidine tag at the N-terminus followed by amino acids 786-1343 of the rat VEGF-R2 kinase domain (GenBank Accession #U93306).

### EXAMPLE 3

### CDK2 Screening Assay

A kinase reaction mixture was prepared containing 50 mM Tris.HCl pH=8, 10 mM MgCl₂, 0.1 mM Na₃PO₄, 1 mM DTT, 10 μM ATP, 0.025 μM biotinylated histone-H1 peptide substrate and 0.2 μCuries per well ³³P-γ-ATP (2000-3000 Ci/mmol). 70 μL of the kinase reaction mixture was dispensed into the well of a streptavidin coated FlashPlate™ (Cat. # SMP103, NEN, Boston, Mass.). Then 1 μL of test compound stock in 100% DMSO was added to the wells resulting in a final concentration of 1% DMSO in the reaction well with a 100 μL final reaction well volume.

The CDK2:Cyclin A protein was diluted in 50 mM Tris.HCl pH=8.0, 0.1% BSA at a concentration of 1 ng per μL and 30 μL (30 ng enzyme per test well) was added to each well to initiate the reaction. The reaction was incubated for one hour at 30° C. At the end of the one hour incubation, the reaction was terminated by aspirating the mixture from the plate and washing the wells twice with PBS containing 100 mM EDTA. The histone-H1 biotinylated peptide substrate became immobilized on the Flashplate™ and the incorporation of ³³P-γ-ATP was measured by reading the plate on a scintillation counter. Inhibition of the enzymatic activity of CDK2 was measured by observing a reduced amount of ³³P-γ-ATP incorporated into the immobilized peptide.

The CDK2 kinase used was complexed with cyclin A and is commercially available (Upstate Biotech, Lake Placid, N.Y.).

Peptide Substrates

| Kinase | Substrate |
|---|---|
| VEGF-R2 | (Biotin)KHKKLAEGSAYEEV-Amide |
| CDK1 | (Biotin)KTPKKAKKPKTPKKAKKL-Amide |
| CDK2 | (Biotin)KTPKKAKKPKTPKKAKKL-Amide |

Results of assays performed on compounds described above are provided below in Table 1. An IC₅₀ listed as >0.1, >1, >10 or >100 means no observed 50% inhibition at the indicated test concentration. An IC₅₀ listed as ˜1, ˜10 or ˜100 means approximately 50% inhibition was observed at the indicated test concentration. ND means the compound was not tested in the assay specified.

TABLE 1
Kinase IC₅₀ (μM)

| Cpd | CDK1 | CDK2 | VEGFR |
|---|---|---|---|
| 1 | >100 | >100 | ~100 |
| 2 | >100 | >100 | >100 |
| 3 | >100 | >100 | >100 |
| 4 | >100 | >100 | >100 |
| 5 | >100 | >100 | >100 |
| 6 | >100 | 5.761 | >100 |
| 7 | >100 | 17.4 | >100 |
| 8 | 17.51 | 3.739 | >100 |
| 9 | >100 | 17.54 | >100 |
| 10 | 3.636 | 0.6497 | 5.96 |
| 11 | >100 | >10 | >100 |
| 12 | >100 | >100 | >100 |
| 13 | >10 | 3.338 | >100 |
| 14 | >100 | 3.086 | >100 |
| 15 | >100 | 89.09 | >100 |
| 16 | >100 | >10 | >100 |
| 17 | >100 | 3.048 | >100 |
| 18 | >100 | 7.513 | >100 |
| 19 | >100 | 10.84 | >100 |
| 20 | >100 | 5.278 | >100 |
| 21 | 14.75 | 1.54 | >100 |
| 22 | 1.313 | 0.2199 | >100 |
| 23 | >10 | 2.125 | >100 |
| 24 | 53.27 | 8.278 | >100 |
| 25 | 36.35 | 6.222 | >100 |
| 26 | >100 | 3.668 | >100 |
| 27 | >100 | 11.95 | >100 |
| 28 | 12.35 | 1.652 | >100 |
| 29 | >100 | 11.57 | >100 |
| 30 | 11.27 | 1.936 | >100 |
| 31 | 55.29 | 3.035 | >100 |
| 32 | >100 | 6.266 | >100 |
| 33 | >100 | >100 | >100 |
| 34 | >100 | 13.6 | >100 |
| 35 | >100 | >100 | >100 |
| 36 | 42.73 | 4.02 | 100 |
| 37 | >100 | 8.016 | >100 |
| 38 | >100 | 11.01 | >100 |
| 39 | 7.147 | 1.033 | >100 |
| 40 | >100 | >100 | >100 |
| 41 | >100 | 2.05 | 100 |
| 42 | >100 | >100 | >100 |
| 43 | >100 | >100 | >100 |
| 44 | 1.667 | 0.3095 | >100 |
| 45 | >10 | 1.665 | >100 |
| 46 | >100 | 16.13 | >100 |
| 47 | 4.299 | 0.6111 | >100 |
| 48 | 1.554 | 0.1557 | 4.583 |
| 49 | 2.706 | 0.6789 | 100 |
| 50 | >100 | >100 | >100 |
| 51 | >100 | 1.188 | >100 |
| 52 | >100 | 1.371 | >100 |
| 53 | >100 | >100 | >100 |
| 54 | >100 | >100 | >100 |
| 55 | >100 | >100 | >100 |
| 56 | >100 | >10 | >100 |
| 57 | >100 | 13.33 | >100 |
| 58 | >100 | >100 | >100 |
| 59 | >100 | >100 | >100 |
| 60 | >100 | >100 | >100 |
| 61 | >100 | >100 | >100 |
| 62 | >100 | >100 | >100 |

### EXAMPLE 4

### Assay to Measure Inhibition of Cell Proliferation

The ability of a test compound to inhibit the proliferation of cell growth was determined by measuring incorporation of ¹⁴C-labelled thymidine into newly synthesized DNA within the cells. This method was used on American Type Culture Collection (ATCC, Virginia) cell lines derived from carcinomas originating from several tissues such as HeLa cervical adenocarcinoma (ATCC Cat. #CCL-2), A375 malignant melanoma (ATCC Cat. #CRL-1619) and HCT-116 colon carcinoma (ATCC Cat. #CCL-247).

The carcinoma cells were trypsinized and counted. The cells (3000-8000 count) were added to each well of a 96-well CytoStar tissue culture treated scintillating microplate (Amersham #RPNQ0160) in complete medium (100 μL) and the plate was incubated in complete medium for 24 hrs at 37° C. in an inert atmosphere containing 5% CO₂.

Test compound (1 μL) in 100% DMSO was added to the plate test-wells with DMSO only added to control-wells. The plate is incubated in complete medium for a second 24 hr period at 37° C. in an atmosphere containing 5% CO₂.

An aliquot of a solution of Methyl ¹⁴C-thymidine (56 mCi/mmol) (NEN #NEC568 or Amersham #CFA532) in complete medium (20 uL to provide 0.2 MCi/well) was also added to each well and the plate was incubated for a third 24 hr period at 37° C. in an atmosphere containing 5% CO₂.

The plate contents were discarded and the plate was washed twice with PBS (200 μL), then PBS (200 μL) was added to each well. The plate was sealed and the degree of methyl ¹⁴C-thymidine incorporation is quantified on a Packard Top Count.

The IC₅₀ values for the compounds tested in various cell lines are shown in Table 2. An IC₅₀ value shown as >10 or >100 means that 50% inhibition was not observed at the highest concentration.

TABLE 2
Cell Proliferation IC₅₀ (μM)

| Cpd | HeLa | HCT116 | A375 |
|---|---|---|---|
| 1 | >10 | ND | ND |
| 2 | >100 | ND | ND |
| 3 | >10 | ND | ND |
| 4 | >10 | ND | ND |
| 5 | >10 | ND | ND |
| 6 | 72.7 | ND | ND |
| 48 | 20.61 | 5.038 | 10.43 |

### EXAMPLE 5

### In Vivo Models

### Inhibition of Tumor Growth

The in vivo effect of a compound on the growth of human tumor cells can be evaluated by implanting human tumor cells into the hindflank of athymic mice and administering test compound to the mice. Human tumor cells originating from a variety of different tumor types, such as A375 human melanoma cells, are implanted subcutaneously into the hindflank of male athymic mice (Charles River) and allowed to establish a sizeable tumor for 6-10 days as determined by caliper measurements.

A test compound is then administered by injecting the compound formulated in an appropriate vehicle intraperitoneally into the mice once a day for 30 days. The test compound can also be administered by other routes such as orally, sub cutaneously or by intravenous infusion. The size of the tumor in this study is measured every four days and the degree of inhibition is determined by comparing drug-treated animals to animals that are injected with vehicle only.

The synergistic action or enhancement of conventional chemotherapeutic agent by a test compound can also be determined with this model by comparing animals treated with the standard therapy alone to animals treated with test compound plus the same standard therapy. An additive effect on the delay of tumor growth will be observed if synergistic action due to test compound is occurring.

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. These publications are hereby incorporated by reference in their entirety into this application to describe more fully the state of the art to which this invention pertains.

What is claimed is:

1. A compound of formula (I) 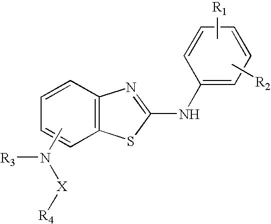
and forms thereof, wherein
R₁ is hydrogen or is selected from C₁₋₆alkyl, C₁₋₆alkoxy, amino, halogen, cyano, amino-sulfonyl, C₁₋₄alkyl-amino-sulfonyl, halo-C₁₋₄alkyl or halo-C₁₋₄alkoxy;
R₂ is hydrogen or is selected from aryl, heteroaryl, heterocyclyl or C₃₋₁₂cycloalkyl optionally substituted with one or two substituents selected from C₁₋₆alkyl, C₁₋₆alkoxy or halogen;
R₃ is hydrogen or is C₁₋₄alkyl;
X is selected from carbonyl, amino-carbonyl, oxy-carbonyl or sulfonyl; and
R₄ is selected from a C₁₋₄alkyl, aryl, aryl-C₁₋₄alkyl, heteroaryl, heteroaryl-C₁₋₄alkyl, heterocyclyl, heterocyclyl-C₁₋₄alkyl, C₃₋₁₂cycloalkyl or C₃₋₁₂cycloalkyl-C₁₋₄alkyl optionally substituted on aryl, heteroaryl, heterocyclyl or C₃₋₁₂cycloalkyl with one, two or three substituents selected from C₁₋₆alkyl, C₁₋₆alkenyl, C₁₋₆alkynyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl, heteroaryl, heterocyclyl or C₃₋₁₂cycloalkyl.

2. The compound of claim 1, wherein R₁ is hydrogen or is selected from C₁₋₆alkyl, C₁₋₆alkoxy or amino-sulfonyl.

3. The compound of claim 1, wherein R₂ is hydrogen or is selected from aryl, heteroaryl, heterocyclyl or C₃₋₁₂cycloalkyl optionally substituted with one substituent selected from C₁₋₆alkyl, C₁₋₆alkoxy or halogen.

4. The compound of claim 1, wherein R₂ is piperazinyl optionally substituted with C₁₋₆alkyl.

5. The compound of claim 1, wherein R₄ is selected from a C₁₋₄alkyl, aryl, aryl-C₁₋₄alkyl, heteroaryl, heteroaryl-C₁₋₄alkyl, heterocyclyl, C₃₋₁₂cycloalkyl or C₃₋₁₂cycloalkyl-C₁₋₄alkyl optionally substituted on aryl, heteroaryl or heterocyclyl with one, two or three substituents selected from C₁₋₆alkyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl or heterocyclyl.

6. The compound of claim 1, wherein R₄ is selected from a C₁₋₄alkyl, phenyl, naphthyl, phenyl-C₁₋₄alkyl, thienyl, furanyl, pyrazolyl, isoxazolyl, [1,2,3]thiadiazolyl, pyridinyl, pyrimidinyl, benzothienyl, indolyl, tetrazolyl-C₁₋₄alkyl, morpholinyl, piperidinyl, piperazinyl, benzo[1,3]dioxolyl, cyclopenyl, cyclohexyl, cycloheptyl or cyclohexyl-C₁₋₄-alkyl optionally substituted on phenyl, thienyl, pyrazolyl, [1,2,3]thiadiazolyl, pyridinyl, indolyl, piperidinyl with one, two or three substituents selected from C₁₋₆alkyl, C₁₋₆alkoxy, C₁₋₆alkyl-carbonyl, amino, halogen, cyano, nitro, amino-sulfonyl, halo-C₁₋₄alkyl, phenyl or morpholinyl.

7. The compound of claim 1, wherein
R₁ is hydrogen or is selected from C₁₋₆alkyl, C₁₋₆alkoxy or amino-sulfonyl;
R₂ is hydrogen or is heterocyclyl optionally substituted with C₁₋₆alkyl;
R₃ is hydrogen or is C₁₋₄alkyl;
X is selected from carbonyl, amino-carbonyl, oxy-carbonyl or sulfonyl; and
R₄ is selected from a C₁₋₄alkyl, aryl, aryl-C₁₋₄alkyl, heteroaryl, heteroaryl-C₁₋₄alkyl, heterocyclyl, C₃₋₁₂cycloalkyl or C₃₋₁₂cycloalkyl-C₁₋₄alkyl optionally substituted on aryl, heteroaryl or heterocyclyl with one, two or three substituents selected from C₁₋₆alkyl, C₁₋₆alkoxy, C₁₋₆alkyl-carbonyl, C₁₋₆alkoxy-carbonyl, amino, halogen, cyano, nitro, amino-sulfonyl, halo-C₁₋₄alkyl, halo-C₁₋₄alkoxy, aryl or heterocyclyl.

8. The compound of claim 1, wherein
R₁ is hydrogen or is selected from C₁₋₆alkyl, C₁₋₆alkoxy or amino-sulfonyl;
R₂ is hydrogen or is heterocyclyl optionally substituted with C₁₋₆alkyl;
R₃ is hydrogen or is C₁₋₄alkyl;
X is selected from carbonyl, amino-carbonyl, oxy-carbonyl or sulfonyl; and
R₄ is selected from a C₁₋₄alkyl, phenyl, naphthyl, phenyl-C₁₋₄alkyl, thienyl, furanyl, pyrazolyl, isoxazolyl, [1,2,3]thiadiazolyl, pyridinyl, pyrimidinyl, benzothienyl, indolyl, tetrazolyl-C₁₋₄alkyl, morpholinyl, piperidinyl, piperazinyl, benzo[1,3]dioxolyl, cyclopenyl, cyclohexyl, cycloheptyl or cyclohexyl-C₁₋₄alkyl optionally substituted on phenyl, thienyl, pyrazolyl, [1,2,3]thiadiazolyl, pyridinyl, indolyl, piperidinyl with one, two or three substituents selected from C₁₋₆alkyl, C₁₋₆alkoxy, C₁₋₆alkyl-carbonyl, amino, halogen, cyano, nitro, amino-sulfonyl, halo-C₁₋₄alkyl, phenyl or morpholinyl.

9. The compound of claim 1, selected from:
1-(2,6-dichloro-phenyl)-3-(2-phenylamino-benzothiazol-4-yl)-urea,
1-(2,6-difluoro-phenyl)-3-(2-phenylamino-benzothiazol-6-yl)-urea,
1-phenyl-3-(2-phenylamino-benzothiazol-7-yl)-urea,
1-(3,4-dichloro-phenyl)-3-(2-phenylamino-benzothiazol-7-yl)-urea,
1-phenyl-3-(2-phenylamino-benzothiazol-4-yl)-urea,
1-(2,6-difluoro-phenyl)-3-(2-phenylamino-benzothiazol-4-yl)-urea,
1-(2-fluoro-phenyl)-3-(2-phenylamino-benzothiazol-4-yl)-urea,
1-(2,6-dichloro-phenyl)-3-(2-phenylamino-benzothiazol-4-yl)-urea,
N-(2-phenylamino-benzothiazol-4-yl)-benzamide,
2,6-difluoro-N-(2-phenylamino-benzothiazol-4-yl)-benzamide,
2,6-difluoro-N-[2-(4-methoxy-phenylamino)-benzothiazol-4-yl]-3-methyl-benzamide,
2,6-difluoro-3-methyl-N-[2-(4-sulfamoyl-phenylamino)-benzothiazol-4-yl]-benzamide,
2,5-dimethyl-2H-pyrazole-3-carboxylic acid (2-phenylamino-benzothiazol-4-yl)-amide,
2,6-difluoro-3-methyl-N-(2-p-tolylamino-benzothiazol-4-yl)-benzamide, pyrimidine-4-carboxylic acid (2-phenylamino-benzothiazol-4-yl)-amide,
N-(2-phenylamino-benzothiazol-4-yl)-acetamide,
N-(2-phenylamino-benzothiazol-4-yl)-4-sulfamoyl-benzamide, isoxazole-5-carboxylic acid (2-phenylamino-benzothiazol-4-yl)-amide,
3-nitro-N-(2-phenylamino-benzothiazol-4-yl)-benzamide,
3-amino-N-(2-phenylamino-benzothiazol-4-yl)-benzamide,
6-morpholin-4-yl-N-(2-phenylamino-benzothiazol-4-yl)-nicotinamide,
N-(2-phenylamino-benzothiazol-4-yl)-2-tetrazol-1-yl-acetamide,
2-(3,5-difluoro-phenyl)-N-(2-phenylamino-benzothiazol-4-yl)-acetamide,
5-bromo-N-(2-phenylamino-benzothiazol-4-yl)-nicotinamide,
1-acetyl-piperidine-4-carboxylic acid (2-phenylamino-benzothiazol-4-yl)-amide,
N-(2-phenylamino-benzothiazol-4-yl)-propionamide, pyridine-2-carboxylic acid (2-phenylamino-benzothiazol-4-yl)-amide,
N-(2-phenylamino-benzothiazol-4-yl)-isonicotinamide,
benzo[1,3]dioxole-5-carboxylic acid (2-phenylamino-benzothiazol-4-yl)-amide,
N-(2-phenylamino-benzothiazol-4-yl)-benzenesulfonamide,
N-(2-phenylamino-benzothiazol-4-yl)-3,5-bis-trifluoromethyl-benzamide,
2-bromo-N-(2-phenylamino-benzothiazol-4-yl)-benzamide,
3-bromo-N-(2-phenylamino-benzothiazol-4-yl)-benzamide,
biphenyl-4-carboxylic acid (2-phenylamino-benzothiazol-4-yl)-amide, thiophene-2-carboxylic acid (2-phenylamino-benzothiazol-4-yl)-amide,
N-(2-phenylamino-benzothiazol-4-yl)-nicotinamide, furan-2-carboxylic acid (2-phenylamino-benzothiazol-4-yl)-amide,
3-cyano-N-(2-phenylamino-benzothiazol-4-yl)-benzamide,
3,5-dimethyl-N-(2-phenylamino-benzothiazol-4-yl)-benzamide,
naphthalene-2-carboxylic acid (2-phenylamino-benzothiazol-4-yl)-amide,
cyclohexanecarboxylic acid (2-phenylamino-benzothiazol-4-yl)-amide,
5-ethyl-thiophene-2-carboxylic acid (2-phenylamino-benzothiazol-4-yl)-amide,
3,5-dinitro-N-(2-phenylamino-benzothiazol-4-yl)-benzamide,
2,4,6-trichloro-N-(2-phenylamino-benzothiazol-4-yl)-benzamide,
2,4,6-trifluoro-N-(2-phenylamino-benzothiazol-4-yl)-benzamide,
2,6-dimethoxy-N-(2-phenylamino-benzothiazol-4-yl)-benzamide,
2,6-dichloro-N-(2-phenylamino-benzothiazol-4-yl)-benzamide,
2,6-difluoro-3-methyl-N-(2-phenylamino-benzothiazol-4-yl)-benzamide,
2,6-difluoro-3-methyl-N-{2-[4-(4-methyl-piperazin-1-yl)-phenylamino]-benzothiazol-4-yl}-benzamide,
benzo[b]thiophene-2-carboxylic acid (2-phenylamino-benzothiazol-4-yl)-amide,
2-phenyl-N-(2-phenylamino-benzothiazol-4-yl)-acetamide,
cyclopentanecarboxylic acid (2-phenylamino-benzothiazol-4-yl)-amide,
(2-phenylamino-benzothiazol-4-yl)-carbamic acid phenyl ester,
3-phenyl-N-(2-phenylamino-benzothiazol-4-yl)-propionamide, cycloheptanecarboxylic acid (2-phenylamino-benzothiazol-4-yl)-amide,
4-methyl-[1,2,3]thiadiazole-5-carboxylic acid (2-phenylamino-benzothiazol-4-yl)-amide,
2,2-dimethyl-N-(2-phenylamino-benzothiazol-4-yl)-propionamide,
2-cyclohexyl-N-(2-phenylamino-benzothiazol-4-yl)-acetamide,
4,6-dichloro-1H-indole-2-carboxylic acid (2-phenylamino-benzothiazol-4-yl)-amide, and
1-tert-butyl-3-(2-phenylamino-benzothiazol-4-yl)-urea,
1-cyclohexyl-3-(2-phenylamino-benzothiazol-4-yl)-urea, or
2,6-difluoro-3,N-dimethyl-N-(2-phenylamino-benzothiazol-4-yl)-benzamide.

10. The compound of claim 1, selected from:
1-(2,6-dichloro-phenyl)-3-(2-phenylamino-benzothiazol-4-yl)-urea,
2,6-difluoro-N-(2-phenylamino-benzothiazol-4-yl)-benzamide,
2,5-dimethyl-2H-pyrazole-3-carboxylic acid (2-phenylamino-benzothiazol-4-yl)-amide,
2,6-difluoro-3-methyl-N-(2-p-tolylamino-benzothiazol-4-yl)-benzamide,
N-(2-phenylamino-benzothiazol-4-yl)-4-sulfamoyl-benzamide,
6-morpholin-4-yl-N-(2-phenylamino-benzothiazol-4-yl)-nicotinamide,
N-(2-phenylamino-benzothiazol-4-yl)-2-tetrazol-1-yl-acetamide,
2-(3,5-difluoro-phenyl)-N-(2-phenylamino-benzothiazol-4-yl)acetamide,
N-(2-phenylamino-benzothiazol-4-yl)-propionamide,
N-(2-phenylamino-benzothiazol-4-yl)-isonicotinamide,
N-(2-phenylamino-benzothiazol-4-yl)-benzenesulfonamide,
N-(2-phenylamino-benzothiazol-4-yl)-3,5-bis-trifluoromethylbenzamide,
N-(2-phenylamino-benzothiazol-4-yl)-nicotinamide,
3,5-dimethyl-N-(2-phenylamino-benzothiazol-4-yl)-benzamide, cyclohexanecarboxylic acid (2-phenylamino-benzothiazol-4-yl)-amide,
2,4,6-trichloro-N-(2-phenylamino-benzothiazol-4-yl)-benzamide,
2,4,6-trifluoro-N-(2-phenylamino-benzothiazol-4-yl)-benzamide,
2,6-dichloro-N-(2-phenylamino-benzothiazol-4-yl)-benzamide,
2,6-difluoro-3-methyl-N-(2-phenylamino-benzothiazol-4-yl)-benzamide,
2,6-difluoro-3-methyl-N-{2-[4-(4-methyl-piperazin-1-yl)-phenylamino]-benzothiazol-4-yl}-benzamide,
2-phenyl-N-(2-phenylamino-benzothiazol-4-yl)-acetamide, or
cyclopentanecarboxylic acid (2-phenylamino-benzothiazol-4-yl)-amide.

11. An intermediate compound of Formula (Ia): 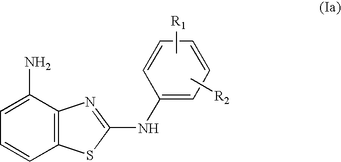
wherein
R₁ is hydrogen or is selected from C₁₋₆alkyl, C₁₋₆alkoxy, amino, halogen, cyano, amino-sulfonyl, C₁₋₄alkyl-amino-sulfonyl, halo-C₁₋₄alkyl or halo-C₁₋₄alkoxy; and,
R₂ is hydrogen or is selected from aryl, heteroaryl, heterocyclyl or C₃₋₁₂cycloalkyl optionally substituted with one or two substituents selected from C₁₋₆alkyl, C₁₋₆alkoxy or halogen.

12. The intermediate of claim 11, wherein R₁ is hydrogen or is selected from C₁₋₆alkyl, C₁₋₆alkoxy or amino-sulfonyl.

13. The intermediate of claim 11, wherein R₂ is hydrogen or is selected from heterocyclyl substituted with one C₁₋₆alkyl substituent.

14. An intermediate compound selected from the group consisting of:
N²-phenyl-benzothiazole-2,4-diamine,
N²-(4-methoxy-phenyl)-benzothiazole-2,4-diamine,
4-(4-amino-benzothiazol-2-ylamino)-benzenesulfonamide, and
N²-p-tolyl-benzothiazole-2,4-diamine.

15. The compound of claim 1, wherein the compound is a CDK or VEGF protein kinase inhibitor.

16. The compound of claim 15, wherein the protein kinase is selected from the group consisting of CDK-1, CDK-2 and VEGF-R2.

17. The compound of claim 1, wherein the compound is an isolated form thereof.

18. A pharmaceutical composition comprising an effective amount of the compound of claim 1.

19. The pharmaceutical composition of claim 18, wherein the effective amount of the compound is in a range of from about 0.001 mg/kg to about 300 mg/kg of body weight per day.

20. A process for preparing a pharmaceutical composition comprising the step of admixing the compound of claim 1 and a pharmaceutically acceptable carrier.

21. A method for treating a chronic or acute protein kinase mediated disease, disorder or condition in a subject in need thereof comprising administering to the subject an effective amount of the compound of claim 1.

22. The method of claim 21, further comprising treating a chronic or acute CDK-1, CDK-2 and VEGF-R2 kinase mediated disease, disorder or condition.

23. The method of claim 21, wherein the effective amount of the compound is in a range of from about 0.001 mg/kg to about 300 mg/kg of body weight per day.

24. The method of claim 21, wherein the disease, disorder or condition is osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohns disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, occular diseases, viral infections, heart disease, lung or pulmonary diseases or kidney or renal diseases.

25. The method of claim 21, wherein acute or chronic cancer is selected from bladder cancer, brain, head or neck cancer, breast cancer, colorectal cancer, endometrial cancer, epidermoid cancer, esophageal cancer, gastric cancer, glioma cancer, lung cancer, ovarian cancer, pancreatic cancer, prostate cancer, renal cell cancer, Kaposi's sarcoma, leukemia, lymphoma or papillocarcinoma; and, cancer-associated pathologies selected from abnormal cell proliferation, unregulated cell proliferation, tumor growth, tumor angiopathy, tumor angiogenesis, tumor vascularization or metastatic cancer cell invasion and migration.

26. The method of claim 21, wherein fibroproliferative and differentiative skin diseases or disorders are selected from papilloma formation, psoriasis, dermatitis, eczema, seborrhea or chemotherapy-induced alopecia; wherein central nervous system diseases are selected from Alzheimer's disease, Parkinson's disease or depression; wherein occular diseases are selected from macular degeneration, diseases of the cornea or glaucoma; wherein viral infections are selected from mycotic infection, autoimmune disease or cytomegalovirus; wherein heart disease is selected from atherosclerosis, neointima formation or transplantation-induced vasculopathies such as arterial restenosis; wherein lung or pulmonary diseases are selected from allergic-asthma, lung fibrosis, pulmonary fibrosis or chronic obstructive pulmonary disorder; and, wherein kidney or renal diseases are selected from acute, subacute or chronic forms of glomerulonephritis or membranoproliferative glomerulonephritis, glomerulosclerosis, congenital multicystic renal dysplasia or kidney fibrosis.

27. A process for preparing the compound of claim 1 comprising the steps of:
Step A. reacting a Compound A1 (wherein Ra is a halogen leaving group) with a strong acid (such as concentrated H₂SO₄, concentrated HNO₃ and the like and mixtures thereof) to provide a Compound A2: 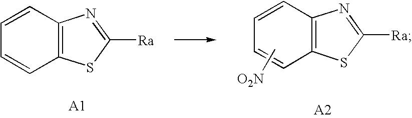
Step B. reacting a solution of Compound A2 (1 equivalent) (in a solvent such as THF, IPA and the like and mixtures thereof) with a Compound A3 (20 equivalents), in the presence of a reagent (2 equivalents) (such as K₂CO₃ and the like) to provide a Compound A4: 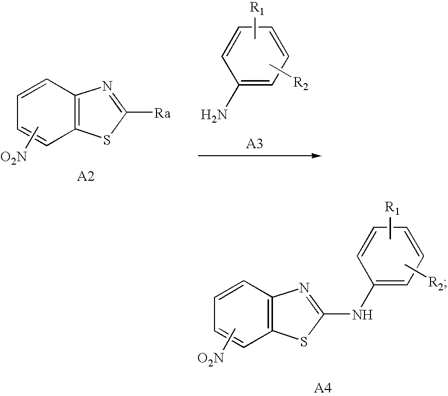
Step C. reacting Compound A4 with a reducing metal (such as iron powder and the like) in the presence of an acid (such as HCl, acetic acid and the like) or by hydrogenation (using hydrogen gas under pressure in the range of from about 30 to about 50 psi) in the presence of a catalyst (such as Raney nickel, palladium on carbon and the like) to provide a Compound A5: 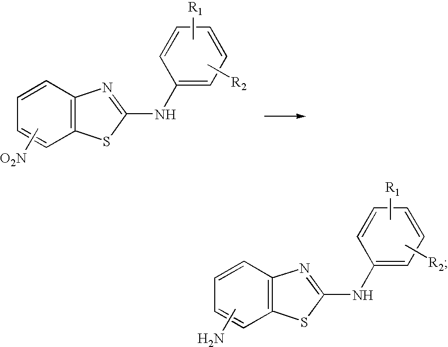
Step D. reacting a solution of Compound A5 (1 equivalent) (in a solvent such as CH₂Cl₂, DMF and the like) with a Compound A6 (1 equivalent) (wherein Xa is a reactive group such as isocyanato, acid chloride, carboxylic acid and the like and wherein certain portions of Xa are incorporated into X as a product of the reaction) in the optional presence of a reagent to provide a Compound A7, representative of a compound of formula (I): 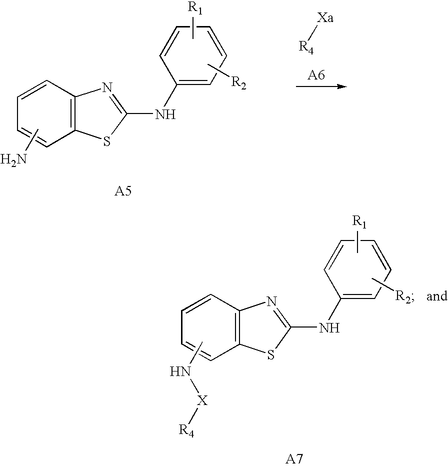
Step E. reacting Compound A7 (in a solvent such as DMF and the like), in the presence of a reagent (such as NaH and the like) with a Compound A8 (wherein Xb is a halogen leaving group) to provide a compound of formula (I): [chemical structure]
